(19) United States
(12) Patent Application Publication
(10) Pub. No.: US 2020/0392075 A1
(43) Pub. Date: Dec. 17, 2020

(54) COMPOSITIONS AND METHODS FOR TREATMENT OF INSULIN RESISTANCE

(71) Applicant: Temple University - Of The Commonwealth System Of Higher Education, Philadelphia, PA (US)

(72) Inventors: Salim Merali, Philadelphia, PA (US); Carlos A. Barrero, Philadelphia, PA (US); Wayne E. Childers, New Hope, PA (US); George C. Morton, Collegeville, PA (US)

(21) Appl. No.: 16/977,676

(22) PCT Filed: Mar. 7, 2019
(86) PCT No.: PCT/US2019/021220

§ 371 Date: Sep. 2, 2020

Related U.S. Application Data
(60) Provisional application No. 62/639,803, filed on Mar. 7, 2018.

(51) Int. Cl. C07C 271/22; C07C 229/22; A61P 3/10

(52) U.S. Cl. CPC C07C 271/22; A61P 3/10; C07C 229/22

(57) ABSTRACT

Compounds of Formula (I), and pharmaceutically acceptable salts thereof, wherein X is selected from the group consisting of —O—, —S—, —NR¹⁴—O—, —O—NR¹⁴—, —NR¹⁴—NR¹⁵— and —S—S—; R¹-R¹⁵, m, n, o, p, q and r are as defined herein, are provided for treatment for increasing insulin sensitivity, reducing insulin resistance, preventing insulin resistance and treating insulin resistance disorders.

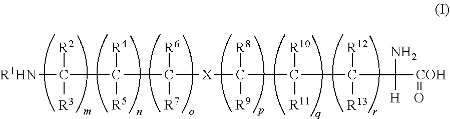

### CROSS-REFERENCE TO RELATED APPLICATION

This application claims the benefit of U.S. Provisional application No. 62/639,803, filed Mar. 7, 2018, the entire disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Mar. 6, 2019 is named 35926_0500_WO_587369_SL.txt and is 1,199 bytes in size.

### FIELD OF THE INVENTION

The invention relates to the field of metabolic diseases. Particularly, the invention relates to treatment of insulin resistance.

### BACKGROUND OF THE INVENTION

In Type 1 diabetes, also known as insulin-dependent diabetes mellitus (IDDM), or juvenile diabetes, the pancreas produces little or no insulin. Type 1 diabetes is believed to result in part from the autoimmune attack on the insulin producing beta-cells of the pancreas.

Type 2 diabetes mellitus (2DM), also known as Non-Insulin Dependent Diabetes Mellitus (NIDDM), or adult-onset diabetes, is mostly caused by insulin resistance and eventually results in beta-cell exhaustion, leading to beta-cell destruction. Insulin resistance is associated with impairment of peripheral tissue response to insulin. T2DM is primarily due to obesity and not insufficient exercise in people who are genetically predisposed. It makes up about 90% of cases of diabetes. Rates of T2DM have increased markedly since 1960 in parallel with obesity. It is believed to afflict approximately 18.2 million people in the US. T2DM typically begins in middle or older age. However, as a result of the obesity epidemic, substantially younger patients are diagnosed with this condition. Type 2 diabetes is associated with a ten-year-shorter life expectancy.

Insulin resistance is generally regarded as a pathological condition in which cells fail to respond to the normal actions of the hormone insulin. When the body produces insulin under conditions of insulin resistance, the cells in the body are resistant to the insulin and are unable to use it as effectively, leading to high blood sugar.

In the early stage of T2DM, the predominant abnormality is reduced insulin sensitivity. At this stage hyperglycemia can be reversed by a variety of measures and medications known in the art. In reaction to increasing insulin resistance, beta-cells are forced to produce more insulin, or are triggered to proliferate and/or granulate, producing even more insulin. The overproduction of insulin or over activity of beta-cells can then lead to beta-cell exhaustion, leading to destruction of the beta-cell population. The pancreas can thus no longer provide adequate levels of insulin, resulting in elevated levels of glucose in the blood. Ultimately, overt hyperglycemia and hyperlipidemia occur, leading to the devastating long-term complications associated with diabetes, including cardiovascular disease, renal failure, and blindness.

Insulin resistance is present in almost all obese individuals (Paoletti et al., Vasc Health Risk Manag 2:145-152). Obesity-linked insulin resistance greatly increases the risk for T2DM, hypertension, dyslipidemia, and nonalcoholic fatty liver disease, together known as the metabolic or insulin resistance syndrome (Reaven, Diabetes, 37: 1595-1607 (1988)).

Insulin resistance and T2DM are associated to increased risk of heart attacks, strokes, amputation, diabetic retinopathy, and kidney failure. For extreme cases, circulation of limbs is affected, potentially requiring amputation. Loss of hearing, eyesight, and cognitive ability has also been linked to these conditions.

Management of insulin resistance in children and adults is essentially based on dietary and lifestyle changes, including healthier dietary habits and increased exercise. These practices can be very efficient in improving insulin sensitivity and in slowing the progression of the disease, but they are difficult to apply and actually not followed by most patients. T2DM can be treated with drugs promoting insulin sensitivity, e.g., thiazolidinedionesthes, but their efficacy in reducing the rate of progression of the disease is quite low. Insulin treatment is required during the most advanced phases of the disease.

Thiazolidinediones, such as troglitazone, rosiglitazone and pioglitazone, bind to peroxisome proliferator-activated receptors, a group of receptor molecules inside the cell nucleus. The normal ligands for these receptors are free fatty acids (FFAs) and eicosanoids. When activated, the receptor migrates to the DNA, activating transcription of a number of specific genes. The activation of these different genes results in 1) decreasing insulin resistance, 2) modifying adipocyte differentiation, 3) inhibiting VEGF-induced angiogenesis, 4) decreasing leptin levels (leading to an increased appetite), 5) decreasing certain interleukins (e.g., IL-6) levels, and 6) increasing adiponectin levels. However, thiazolidinedione intake is usually associated with a weight gain. Efficacy in reducing the rate of disease progression is low. Thus, there is a still a need for more effective therapies for insulin resistance.

How obesity promotes insulin resistance remains incompletely understood, although several potential mechanisms have been proposed. Plasma concentrations of free fatty acids and pro-inflammatory cytokines, endoplasmic reticulum (ER) stress, and oxidative stress are all elevated in obesity and have been shown to induce insulin resistance. However, they may be late events that only develop after chronic excessive nutrient intake.

In overnutrition, excessive glucose is consumed and a large amount of glucose is metabolized via glycolysis and the TCA cycle leading to increased NADH and FADH2 production in the mitochondrial electron transport chain and increased reactive oxygen species (ROS). When the generation of ROS exceeds their detoxification, oxidative stress occurs. Oxidative stress may cause reversible or irreversible changes in proteins. Reversible changes occur in cysteine residues and can be repaired by antioxidant proteins. On the other hand, oxidative stress can directly or indirectly induce irreversible damage to the proteins by formation of reactive carbonyl groups, mainly aldehydes and ketones. Direct protein carbonylation of lysine or arginine residues occurs through a Fenton reaction of metal cations with hydrogen peroxide, forming glutamic semialdehyde. Indirect carbonylation can occur by reactive α,β-unsaturated aldehydes, which are products of oxidative modification of polyunsaturated fatty acids (PUFA).

The most common reactive aldehyde is 4-hydroxynonenal (4-HNE). 4-HNE reacts with cysteine, lysine, and histidine residues of proteins via Michael addition and Schiff base formation. The introduction of carbonyl derivatives (i.e. aldehydes and ketones) alters the conformation of the polypeptide chain, resulting in the partial or total inactivation of proteins. Because protein carbonylation is an irreversible process, it is deleterious to the cells. 4-HNE increases have been reported in T2DM and in the liver of diabetic rats.

In a study reported in 2015, healthy men were fed with ˜6000 kcal/day of the common U.S. diet [˜50% carbohydrate (CHO), ˜35% fat, and ˜15% protein] for 1 week. The diet produced a rapid weight gain of 3.5 kg and the rapid onset (after 2 to 3 days) of systemic and adipose tissue insulin resistance and oxidative stress but no inflammatory or ER stress. Boden et al., Science Translation Medicine, 7 (304): 304re7 (9 Sep. 2015). In adipose tissue, the oxidative stress is associated with several GLUT4 posttranslational modifications, including extensive GLUT4 carbonylation as well as adduction of HNE and glutamic semialdehyde in close proximity to the glucose transport channel. Id GLUT4 is the major insulin-facilitated glucose transporter in adipose tissue. Carbonylation typically causes protein cross-linking and loss or alteration of protein function (Schaur, Mol. Aspects Med 24: 149-159 (2003) and can target the affected proteins for selective degradation by the 26S proteasome (Kastle et al., Curr. Pharm. Des. 17: 4007-4022 (2011)).

Notwithstanding these advances, what is still needed am therapeutic agents for the prevention and treatment of insulin resistance, particularly in obese patients who typically suffer from insulin resistance or are most susceptible to the development of insulin resistance, and ultimately, type 2 diabetes.

### SUMMARY OF THE INVENTION

Provided are compounds according to Formula I, and pharmaceutically acceptable salts thereof:

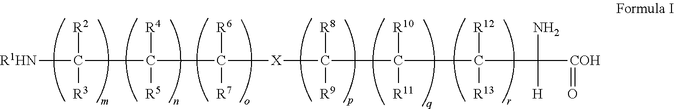

wherein:

X is selected from the group consisting of —O—, —S—, —NR¹⁴—O—, —O—NR¹⁴—, —NR¹⁴—NR¹⁵— and —S—S—;

R¹ is selected from the group consisting of hydrogen, —(C₁-C₈)alkyl, —(C₁-C₈)alkenyl, —(C₁-C₅)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;

R², R³, R⁶, R⁷, R⁸, R⁹, R¹², and R¹³ are independently selected from the group consisting of hydrogen and —(C₁-C₆)alkyl;

R⁴ and R³ are independently selected from the group consisting of hydrogen, —(C₁-C₆)alkyl and —OH, provided that both R⁴ and R⁵ cannot be —OH;

R¹⁰ and R¹¹ are independently selected from the group consisting of hydrogen, —(C₁-C₆)alkyl and —OH, provided that both R¹⁰ and R¹¹ cannot be —OH;

R¹⁴ and R¹⁵ are independently selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;

m is 1, 2 , 3 or 4;

n is 0, 1, 2, 3 or 4;

o is 0, 1, 2, 3 or 4;

p is 1, 2 , 3 or 4;

q is 0, 1, 2, 3 or 4; and

r is 0, 1, 2, 3 or 4.

In an embodiment, a method for increasing insulin sensitivity, reducing insulin resistance and/or preventing insulin resistance in a subject in need thereof comprises administering to the subject an effective amount of a compound according to Formula I, or pharmaceutically acceptable salt thereof.

Also provided is a method of treating insulin resistance in a subject in need thereof comprising administering to the patient an effective amount of a compound according to Formula I, or pharmaceutically acceptable salt thereof.

Also provided is a method of treating an insulin resistance disorder in a subject in need thereof comprising administering to the individual an effective amount of a compound according to Formula I, or pharmaceutically acceptable salt thereof.

Also provided is a method of alleviating an insulin resistance disorder in a subject in need thereof comprising administering to the individual an effective amount of a compound according to Formula I, or pharmaceutically acceptable salt thereof. Insulin resistance disorders include, by way of example and not limitation, diabetes, obesity, metabolic syndrome, insulin resistance, insulin-resistance syndromes, syndrome X, high blood pressure, hypertension, high blood cholesterol, hyperlipidemia, dyslipidemia, atherosclerotic disease, hyperglycemia, hyperinsulinemia, hyperproinsulinemia, impaired glucose tolerance, delayed insulin release, coronary heart disease, angina pectoris, congestive heart failure, stroke, cognitive dysfunction, retinopathy, peripheral neuropathy, nephropathy, glomerulonephritis, glomerulosclerosis, nephrotic syndrome, hypertensive nephrosclerosis, endometrial cancer, breast cancer, prostate cancer, colon cancer, complications of pregnancy, menstrual irregularities, infertility, irregular ovulation, polycystic ovarian syndrome (PCOS), lipodystrophy, cholesterol related disorders, gout, obstructive sleep apnea, osteoarthritis, and osteoporosis.

In certain embodiments, the subject is afflicted with insulin resistance, reduced insulin sensitivity or an insulin resistance disorder. In other embodiments, the patient is at risk of developing these conditions, and a compound of Formula I is administered prophylactically, to delay the onset of such conditions, or reduce the severity when the conditions are experienced.

Also provided are compounds according to Formula I, and pharmaceutically acceptable salts thereof, for use in increasing insulin sensitivity, reducing insulin resistance and/or preventing insulin resistance in a subject. Also provided are compounds according to Formula I, and pharmaceutically acceptable salts thereof, for use in treating insulin resistance in a subject. Also provided are compounds according to Formula I, and pharmaceutically acceptable salts thereof, for use in treating an insulin resistance disorder in a subject. Also provided are compounds according to Formula I, and pharmaceutically acceptable salts thereof, for use in alleviating an insulin resistance disorder in a subject.

Also provided is a medicament for increasing insulin sensitivity, reducing insulin resistance and/or preventing insulin resistance in a subject, containing a compound according to Formula I, or pharmaceutically acceptable salt thereof. Also provided is a medicament for treating insulin resistance in a subject, containing a compound according to Formula I, or pharmaceutically acceptable salt thereof. Also provided is a medicament for in treating an insulin resistance disorder in a subject, containing a compound according to Formula I, or pharmaceutically acceptable salt thereof. Also provided is a medicament for alleviating an insulin resistance disorder in a subject.

In certain embodiments of the aforesaid methods and uses of a compound according to Formula I, or pharmaceutically acceptable salt thereof, the compounds of Formula I are the L-isomer substantially free of the D-isomer.

In certain embodiments of the aforesaid methods and uses of a compound according to Formula I, or pharmaceutically acceptable salt thereof, X═—O—. For X═O, it is preferred that the chiral carbon of the amino acid moiety is in the S-configuration, corresponding to natural L-amino acids. In an embodiment, the compound is (S)-2-amino-3-(3-aminopropoxy)propanoic acid, or a pharmaceutically acceptable salt thereof. A preferred salt thereof is (S)-2-amino-3-(3-aminopropoxy)propanoic acid dihydrochloride.

In certain embodiments of the aforesaid methods and uses of a compound according to Formula I, or pharmaceutically acceptable salt thereof, X═S or —S—S—. It is to be noted that the sulfur changes the R/S designation depending on how far away from the chiral center it is. Accordingly, when p+q+r=1, it is preferred that the chiral carbon of the amino acid moiety is in the R-configuration, corresponding to natural L-amino acids. When p+q+r changes is equal to or greater than 2, it is preferred that the chiral carbon of the amino acid moiety is in the S-configuration, corresponding to natural L-amino acids.

In certain embodiments of the aforesaid methods and uses of a compound according to Formula I, or pharmaceutically acceptable salt thereof, X is selected from —NR¹⁴—O—, —O—NR¹⁴—, or —NR¹⁴—NR¹⁵. In these embodiments, it is preferred that the chiral carbon of the amino acid moiety is in the S-configuration, corresponding to natural L-amino acids.

In certain embodiments of the aforesaid methods and uses of a compound according to Formula I, or pharmaceutically acceptable salt thereof, where X is —O—, when the sum of p+q+r is 1 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 3 or greater.

In certain embodiments of the aforesaid methods and uses of a compound according to Formula I, or pharmaceutically acceptable salt thereof, when X is —O—, when the sum of p+q+r is 2 and the sum of m+n+o is 2, R¹ cannot be ethyl.

In certain embodiments of the aforesaid methods and uses of a compound according to Formula I, or pharmaceutically acceptable salt thereof, where X is —O—, when the sum of p+q+r is 2 and the definition of R, R², R³, R⁴, R⁵, R⁶, R⁷, R⁸, R⁹, R¹⁰, R¹¹, R¹², R¹³ and R¹⁴ are all H, the sum of m+n+o is 3 or greater.

In certain embodiments of the aforesaid methods and uses of a compound according to Formula I, or pharmaceutically acceptable salt thereof, where X is —S—, when the sum of p+q+r is 1 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 5 or greater.

In certain embodiments of the aforesaid methods and uses of a compound according to Formula I, or pharmaceutically acceptable salt thereof, where X is —S—, when the sum of p+q+r is 2 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 4 or greater.

In certain embodiments of the aforesaid methods and uses of a compound according to Formula I, or pharmaceutically acceptable salt thereof, where X is —NR¹⁴—O—, and R¹ and R¹⁴ are hydrogen as shown below, the sum of m+n+o is 2, 3 or 4 and/or the sum of p+q+r is 1, 2 or 3.

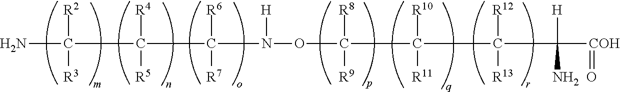

In certain embodiments of the aforesaid methods and uses of a compound according to Formula I, or pharmaceutically acceptable salt thereof, where X is —O—NR¹⁴—, and R¹ and R¹⁴ are hydrogen as shown below, the sum of m+n+o is 2, 3 or 4 and/or the sum of p+q+r is 1, 2, 3 or 4.

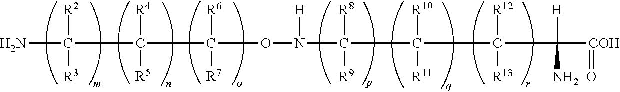

In certain embodiments of the aforesaid methods and uses of a compound according to Formula I, or pharmaceutically acceptable salt thereof, where X is —NR¹⁴—NR¹⁵—, and R¹⁴ and R¹⁵ are hydrogen as shown below, the sum of m+n+o is 2, 3 or 4 and/or the sum of p+q+r is 1, 2, 3 or 4.

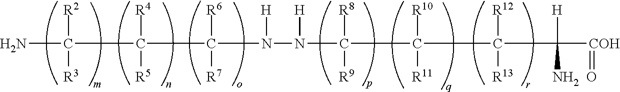

In certain embodiments of the aforesaid methods and uses of a compound according to Formula I, or pharmaceutically acceptable salt thereof, where X is —S—S—, as shown below, the sum of m+n+o is 2, 3 or 4 and/or the sum of p+q+r is 1, 2, 3 or 4.

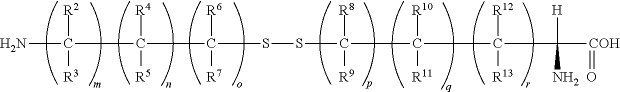

In certain embodiments of the aforesaid methods and uses of a compound according to Formula I, or pharmaceutically acceptable salt thereof, defining sums of m+n+o and/or defining sums of p+q+r, each of R² through R¹³ is independently selected from hydrogen and —(C₁-C₈)alkyl. In certain embodiments, R² through R¹³ are hydrogen.

In certain embodiments of the aforesaid methods and uses of a compound according to Formula I, or pharmaceutically acceptable salt thereof, R¹ is selected from hydrogen and —(C₁-C₈)alkyl. In certain embodiments, R¹⁴ and R¹⁵ are independently selected from hydrogen or —(C₁-C₈)alkyl. In certain embodiments, R¹, R¹⁴, and R¹⁵ are independently selected from hydrogen and —(C₁-C₈)alkyl. In the aforementioned embodiments, the —(C₁-C₈)alkyl is preferably —(C₁-C₆)alkyl, more preferably —(C₁-C₃)alkyl, more preferably methyl or ethyl. In certain embodiments, R¹, R¹⁴, and R¹⁵ are hydrogen.

In certain embodiments of the aforesaid methods and uses of a compound according to Formula I, or pharmaceutically acceptable salt thereof, each of R², R³, R⁴, R⁵, R⁶, and R⁷ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R², R³, R⁴, R⁵, R⁶, and R⁷ are hydrogen.

In certain embodiments of the aforesaid methods and uses of a compound according to Formula I, or pharmaceutically acceptable salt thereof, each of R⁸, R⁹, R¹⁰, R¹¹, R¹², and R¹³ is independently selected from hydrogen and —(C₁-C₃)alkyl. The —(C₁-C₈)alkyl is preferably —(C₁-C₆)alkyl, more preferably —(C₁-C₃)alkyl, more preferably methyl or ethyl. In certain embodiments, R⁸, R⁹, R¹⁰, R¹¹, R¹², and R¹³ are hydrogen.

In certain embodiments of the aforesaid methods and uses of a compound according to Formula I, or pharmaceutically acceptable salt thereof, each of R² through R¹³ are independently selected from hydrogen and —(C₁-C₈)alkyl, according to the above schemes. In certain embodiments, R² through R¹³ are hydrogen.

In an certain embodiments of the aforesaid methods and uses of a compound according to Formula I, or pharmaceutically acceptable salt thereof, the compound of Formula I is (S)-2-amino-3-(3-aminopropoxy)propanoic acid.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### DESCRIPTION OF THE FIGURES

FIG. 1A presents multiple reaction monitoring (MRM) data showing an increase in the transitions of HNE-induced K264-HNE adducts in 3T3-L1 cells overexpressing GLUT4. The cells were treated with 20 μM 4-HNE for 4 hours. The MRM data is then used to calculate the amounts of carbonylated GLUT4.

FIG. 1B shows the carbonylated GLUT4 data calculated from the MRM data in FIG. 1A. Four transitions of the GLUT4 peptide found in humans were used for quantitation.

FIG. 2 shows the effect of 4-HNE and H₂O₂ on insulin-induced glucose uptake by 3T3-L1 adipocytes. Glucose uptake is reduced by 32% and 66% with 4-HNE and H₂O₂ treatment, respectively. The combination of both 4-HNE and H₂O₂ resulted in a 98% decrease in glucose uptake.

FIG. 3A is a plot of the level of GLUT4 carbonylation as determined by detection of the adducted GLUT4 fragment LTGWADVSGVLAELKDEK-4HNE (SEQ ID NO:2), constituting GLUT4 amino acids 247-264 (LTGWADVSGVLAELKDEK, SEQ ID NO: 1), in adipose tissue from lean individuals, and from lean over-nourished insulin-resistant individuals. HP70-1 is utilized as an internal control.

FIG. 3B is a plot of the level GLUT4 carbonylation as determined by detection of the adducted GLUT4 fragment LTGWADVSGVLAELKDEK-4HNE (SEQ ID NO:2), in adipose tissue from obese non-diabetic, obese pre-diabetic, and obese diabetic individuals. HP70-1 is utilized as an internal control.

FIG. 3C shows the percentage of GLUT 4 that is carbonylated in adipose tissue of the obese non-diabetic, obese pre-diabetic, and obese diabetic groups of individuals of FIGS. 3A-3B.

FIG. 3D is a graph of GLUT4 carbonylation as determined by detection of the adducted GLUT4 fragment LTGWADVSGVLAELKDEK-4HNE (SEQ ID NO:2) versus the level of the insulin resistance marker HOMA-IR.

### DEFINITIONS

Unless defined otherwise, all technical and scientific terms used herein have the same meaning as commonly understood by one of ordinary skill in the art to which the invention pertains.

Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

The term “alkyl”, by itself or as part of another substituent means, unless otherwise stated, a straight or branched chain hydrocarbyl having the designated number of carbon atoms (i.e., C₁-C₆ means one to six carbons). Examples include: methyl, ethyl, propyl, isopropyl, butyl, isobutyl, tert-butyl, pentyl, neopentyl, and hexyl. Most preferred is (C₁-C₆)alkyl, more preferably (C₁-C₃)alkyl, particularly methyl and ethyl.

The term “alkenyl” employed alone or in combination with other terms, means, unless otherwise stated, a straight chain or branched chain hydrocarbyl having the stated number of carbon atoms, and containing one or more double bonds. Examples include ethenyl (vinyl), propenyl (allyl), crotyl, isopentenyl, butadienyl, 1,3-pentadienyl, and 1,4-pentadienyl. A functional group representing an alkenyl is exemplified by —CH₂—CH═CH₂—.

The term “alkynyl” employed alone or in combination with other terms, means, unless otherwise stated, a straight chain or branched chain hydrocarbyl having the stated number of carbon atoms, and containing on or more triple bonds.

The term “alkoxy” employed alone or in combination with other terms means, unless otherwise stated, an alkyl group, as defined above, connected to the rest of the molecule via an oxygen atom, such as, for example, methoxy, ethoxy, 1-propoxy, 2-propoxy (isopropoxy) and the higher homologs and isomers. The alkyl portion of the alkoxy group can have a designated number of carbon atoms as defined for alkyl groups above. Preferred are (C₁-C₆)alkoxy, more preferably (C₁-C₃)alkoxy, particularly methoxy and ethoxy.

The term “aromatic” refers to a carbocycle or heterocycle having one or more polyunsaturated rings having aromatic character (i.e. having (4n+2) delocalized π (pi) electrons where n is an integer).

The term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “aralkyl” group refers to an alkyl group substituted with an aryl group. For example, the term “ara(c1-c6)alkyl” would be an alkyl group with 1 to 6 carbon atoms substituted with an aryl group.

An “effective amount” as used herein, means an amount which provides the indicated therapeutic or prophylactic benefit, i.e., an amount that results in the treatment and/or prevention of insulin resistance and/or an increase in insulin sensitivity, or treatment and/or prevention if insulin resistance disorder. It is understood, however, that the full therapeutic effect does not necessarily occur by administration of one dose, and may occur only after administration of a series of doses. Thus, an effective amount may be administered in one or more administrations. In the context of therapeutic or prophylactic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. The compounds of Formula I can also be administered in combination with one or more additional therapeutic compounds.

The terms “halo” or “halogen” by themselves or as part of another substituent mean, unless otherwise stated, a fluorine, chlorine, bromine, or iodine atom. Preferably, a halogen includes fluorine, chlorine, or bromine, more preferably, fluorine or chlorine.

The term “heteroaralkyl” group refers to an alkyl group substituted with a heteroaryl group. For instance, the term “heteroara(C₁-C₆)alkyl” would bean alkyl group with 1 to 6 carbon atoms substituted with a heteroaryl group.

The term “heterocycle” or “heterocyclyl” or “heterocyclic” by itself or as part of another substituent means, unless otherwise stated, an unsubstituted or substituted, mono- or multi-cyclic heterocyclic ring system which consists of carbon atoms and at least one heteroatom selected from the group consisting of N, O, and S. The heterocycle typically contains from five to ten ring atoms. The heterocyclic system may be attached to another atom, unless otherwise stated, at any heteroatom or carbon atom of the heterocyclic system which affords a structural isomer.

The term “heteroaryl” or “heteroaromatic” refers to a heterocycle having aromatic character.

The term “hydrocarbyl”, by itself or as part of another substituent means, unless otherwise stated, a straight or branched chain hydrocarbon having the number of carbon atoms designated (i.e. C₁-C₆ means one to six carbons). Examples include: methyl, ethyl, propyl, isopropyl, butyl, isobutyl, tert-butyl, pentyl, neopentyl, and hexyl. Most preferred is (C₁-C₆) alkyl, more preferably (C₁-C₃) particularly methyl and ethyl. The term “unsaturated hydrocarbyl” means a hydrocarbyl that contains at least one double or triple bond.

As used herein, “individual” or “patient” or “subject” (as in the subject of the treatment) means both mammals and non-mammals. Mammals include, for example, humans; non-human primates, e.g. apes and monkeys; dogs; cats; cattle; horses; sheep; and goats. Non-mammals include, for example, fish and birds. The individual is, in one embodiment, a human being. In another embodiment, the individual is a dog.

The term “insulin resistance” has its common meaning in the art. Insulin resistance is a physiological condition where the natural hormone insulin becomes less effective at lowering blood sugars. The resulting increase in blood glucose may raise levels outside the normal range and cause adverse health effects such as metabolic syndrome, dyslipidemia and subsequently type 2 diabetes mellitus.

An “insulin resistance disorder” refers to any disease or condition that is caused by or contributed to by insulin resistance.

The term “haloalkyl” means an alkyl group wherein at least one hydrogen atom is replaced by a halogen atom. The term “perhaloalkyl” means a haloalkyl group wherein all the hydrogen atoms are replaced by halogen atoms. A preferred perhaloalkyl is perfluoroalkyl, particularly —(C₁-C₆)perfluoroalkyl; more preferred is —(C₁-C₃)perfluoroalkyl; most preferred is —CF₃.

The term “haloalkoxy” means an alkoxy group wherein at least one hydrogen atom is replaced by a halogen atom. The term “perhaloalkoxy” means a haloalkoxy group wherein all the hydrogen atoms are replaced by halogen atoms. A preferred perhaloalkoxy is perfluoroalkoxy, particularly —(C₁-C₆)perfluoroalkoxy; more preferred is —(C₁-C₃)perfluoroalkoxy; most preferred is —OCF₃.

As used herein, the term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. Substituents may include, for example, one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, alkylsilyl, cycloalkyl, cycloalkylalkyl, heterocycloalkyl, heteroaryl, alkenyl, alkynyl, aryl, and amino groups. Substituents comprising carbon chains preferably contain 1-6, more preferably 1-3, most preferably 1-2, carbon atoms.

To “treat” a disease as the term is used herein, means to reduce the frequency or severity of at least one sign or symptom of a disease or disorder experienced by a subject. Treating may include the postponement of further disease progression, or reduction in the severity of symptoms that have or are expected to develop, ameliorating existing symptoms and preventing additional symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION OF THE INVENTION

Embodiments of the present invention are described below. It is, however, expressly noted that the present invention is not limited to these embodiments, but rather the intention is that modifications that are apparent to the person skilled in the art and equivalents thereof are also included.

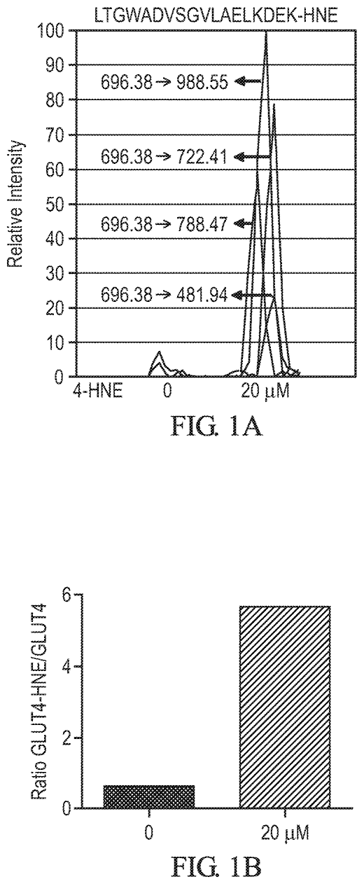

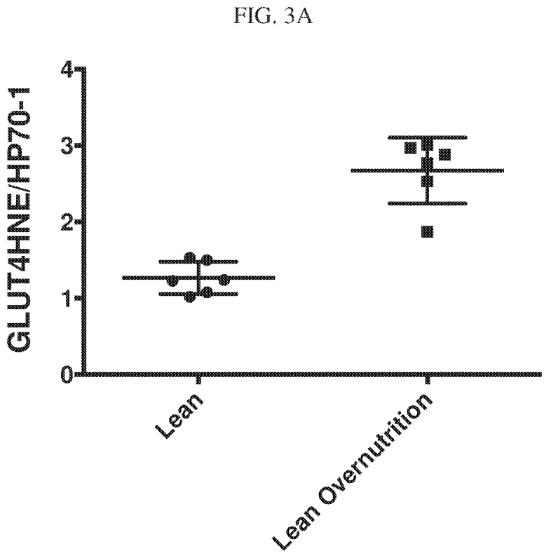

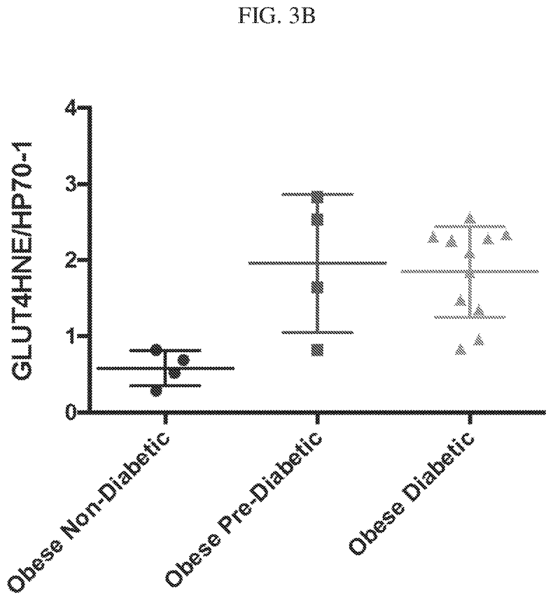

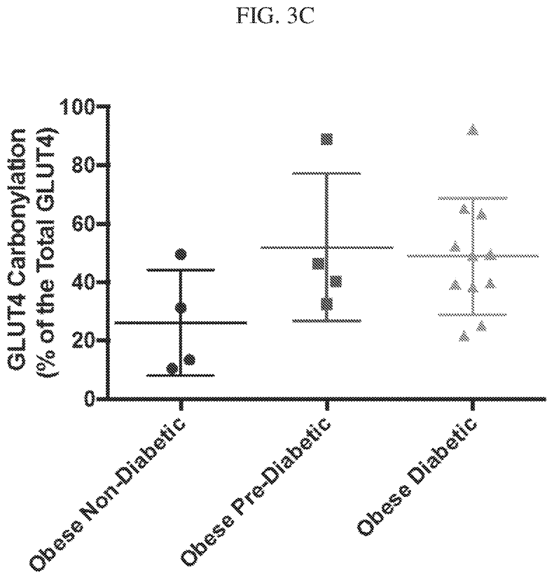

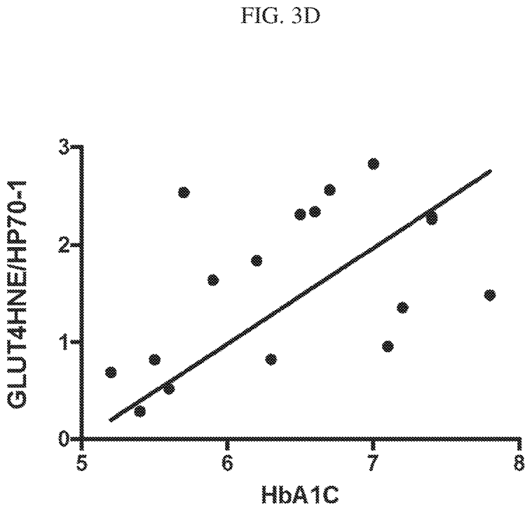

Certain conditions such as overnutrition can lead to oxidative stress, and the generation of reactive aldehydes such as 4-HNE, which react with cysteine, lysine and histidine residues of proteins via Michael addition and Schiff base formation. 4-HNE can form HNE-Michaels adducts on GLUT4, the major insulin-facilitated glucose transporter in adipose tissue. As shown in FIG. 1A, 3T3-L1 adipocytes retrovirally transduced to overexpress the GLUT4-SNAP protein formed a K264-HNE GLUT4 adduct upon treatment with 4-HNE. The amounts of carbonylated protein is shown in FIG. 1B. The same K264-HNE GLUT4 adduct is elevated in the fat tissue of human pre-diabetic and diabetic individuals (FIG. 3B, 3C).

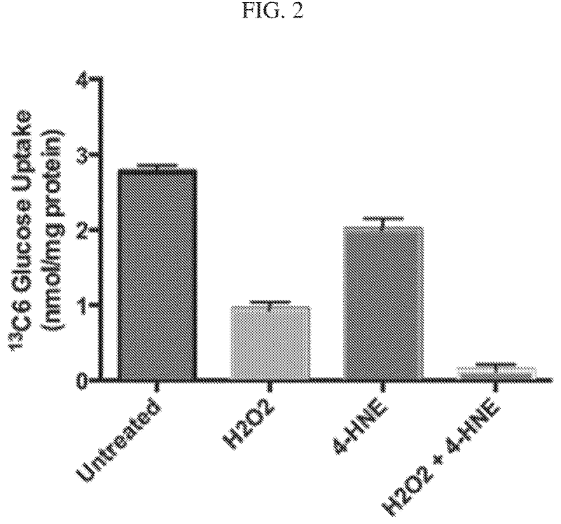

HNE-adduction leads to loss of GLUT-4 function, and development of adipocyte insulin resistance, as indicated by the reduction of adipocyte glucose uptake upon insulin stimulation. As shown in FIG. 2, glucose uptake by 3T3-L adipocytes is reduced upon 4-HNE and H₂O₂ treatment, followed by insulin stimulation.

Compounds of Formula I are believed to be effective in increasing insulin sensitivity and/or reducing insulin resistance.

Compounds of Formula I are believed to overcome adipocyte glucose uptake impairment by restoring insulin sensitivity. Without wishing to be bound by any theory, compounds of Formula I are believed to form adducts with reactive aldehydes such as 4-HNE, thereby diverting 4-HNE from damaging proteins such as GLUT-4, by carbonylation. Formation of an adduct with 4-HNE, thereby diverts 4-HNE from damaging GLUT-4 by carbonylation. It has the effect of reversing overnutrition-induced glucose uptake impairment. Restoration of GLUT-4 function results in enhancement or restoration of adipocyte insulin sensitivity and the resumption or enhancement of glucose uptake.

Improvement of impaired glucose tolerance in comparison to the only moderate glucose tolerance-improving effect of, for instance, pioglitazone can be measured by glucose tolerance tests. Reduction of impaired glucose tolerance relative to metformin, a first-line medication for the treatment of type 2 diabetes, can be demonstrated, for instance, in an animal model of insulin resistance, such as a leptin receptor knock-out mouse.

Compounds that reduce impaired glucose tolerance in both pre-diabetes and diabetes stages, are believed to be therapeutic in both pre-diabetes, and diabetes wherein the diabetic phenotype has been established.

Studies of delay of disease progression in, for instance, an animal model, can demonstrate that compounds are diabetes-modulating, and do not merely mask the disease.

Compounds of Formula I can be used to treat both pre-diabetes, and diabetes wherein the diabetic phenotype has been established. The compounds are believed effective in counteracting glucose uptake impairment in cells induced by overnutrition.

The compounds of Formula i are administered to increase insulin sensitivity and/or reduce insulin resistance in subjects in need of such treatment.

Insulin is produced in the body upon the initiation of glucose release into the bloodstream from carbohydrate digestion. Under normal circumstances, the cells of the body respond to stimulus by insulin by taking up glucose, for use as energy. The major cell types that require insulin to absorb glucose are fat cells and muscle cells. When the body produces insulin under conditions of insulin resistance, these cells in the body are resistant to stimulation by insulin, leading to high blood sugar. Beta cells in the pancreas increase their production of insulin, further contributing to a high blood insulin level. Elevated blood insulin level, left may lead to reduced insulin sensitivity. T2DM, in particular, develops from insulin resistance, meaning that the normally secreted dose of insulin is no longer sufficient to control blood glucose levels.

According to the present invention, any of the pathologies flowing from reduced insulin sensitivity (or insulin resistance) may be treated. The compounds of Formula I are thus useful for treating any condition associated with the loss of the relevant target cell's sensitivity to regulation by insulin. The compounds of Formula I are thus believed useful in the treatment of insulin resistance disorders. An “insulin resistance disorder” refers to refers to any disease or condition that is caused by or contributed to by insulin resistance. Examples include: diabetes, type 2 diabetes (T2DM), pre-diabetes, obesity, metabolic syndrome, insulin resistance, insulin-resistance syndromes, syndrome X, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

In addition to pathological conditions associated with insulin resistance, the compounds of Formula I may be administered for treatment of conditions of low insulin production, e.g. cases of IDDM where some finite level of insulin production remains, albeit at reduced amounts.

### Compounds

Novel compounds and pharmaceutically acceptable salts thereof are provided according to Formula I:

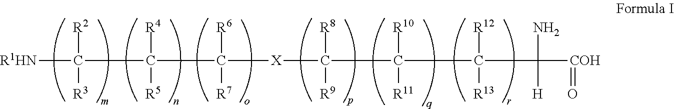

wherein:

X is selected from the group consisting of —O—, —S—, —NR¹⁴—O—, —O—NR¹⁴—, —NR¹⁴—NR¹⁵— and —S—S—;

R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;

R², R³, R⁶, R⁷, R⁸, R⁹, R¹², and R¹³ are independently selected from the group consisting of hydrogen and —(C₁-C₆)alkyl;

R⁴ and R⁵ are independently selected from the group consisting of hydrogen, —(C₁-C₆)alkyl and —OH, provided that both R⁴ and R⁵ cannot be —OH;

R¹⁰ and R¹¹ are independently selected from the group consisting of hydrogen, —(C₁-C₆)alkyl and —OH, provided that both R¹⁰ and R¹¹ cannot be —OH;

R¹⁴ and R¹⁵ are independently selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
m is 1, 2, 3 or 4;
n is 0, 1, 2, 3 or 4;
o is 0, 1, 2, 3 or 4;
p is 1, 2 , 3 or 4;
q is 0, 1, 2, 3 or 4; and
r is 0, 1, 2, 3 or 4.

In certain embodiments, the compounds of Formula I are the L-isomer substantially free of the D-isomer.

In certain embodiments, X═—O—. For X═O, it is preferred that the chiral carbon of the amino acid moiety is in the S-configuration, corresponding to natural L-amino acids. In an embodiment, the compound is (S)-2-amino-3-(3-aminopropoxy)propanoic acid, or a pharmaceutically acceptable salt thereof. A preferred salt thereof is (S)-2-amino-3-(3-aminopropoxy)propanoic acid dihydrochloride.

In certain embodiments, X═S or —S—S—. It is to be noted that the sulfur changes the R/S designation depending on how far away from the chiral center it is. Accordingly, when p+q+r=1, it is preferred that the chiral carbon of the amino acid moiety is in the R-configuration, corresponding to natural L-amino acids. When p+q+r changes is equal to or greater than 2, it is preferred that the chiral carbon of the amino acid moiety is in the S-configuration, corresponding to natural L-amino acids.

In certain embodiments, X is selected from —NR¹⁴—O—, —NR¹⁴—, or —NR¹⁴—NR¹⁵. In these embodiments, it is preferred that the chiral carbon of the amino acid moiety is in the S-configuration, corresponding to natural L-amino acids.

In certain embodiments where X is —O—, when the sum of p+q+r is 1 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 3 or greater.

In certain embodiments when X is —O—, when the sum of p+q+r is 2 and the sum of m+n+o is 2, R¹ cannot be ethyl.

In certain embodiments where X is —O—, when the sum of p+q+r is 2 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 3 or greater.

In certain embodiments, where X is —S—, when the sum of p+q+r is 1 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 5 or greater.

In certain embodiments, where X is —S—, when the sum of p+q+r is 2 and the definition of R¹, R², R³, R⁴, R⁵, R⁶, R⁷, R⁸, R⁹, R¹⁰, R¹¹, R¹², R¹³ and R¹⁴ are all H, the sum of m+n+o is 4 or greater.

In certain embodiments, where X is —NR¹⁴—O—, and R¹ and R¹⁴ are hydrogen as shown below, the sum of m+n+o is 2, 3 or 4 and/or the sum of p+q+r is 1, 2 or 3.

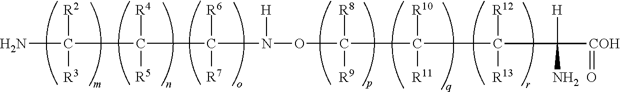

In certain embodiments, where X is —O—NR¹⁴—, and R¹ and R¹⁴ are hydrogen as shown below, the sum of m+n+o is 2, 3 or 4 and/or the sum of p+q+r is 1, 2, 3 or 4.

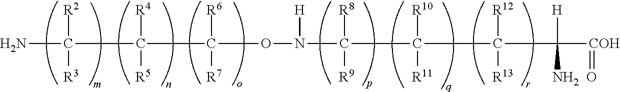

In certain embodiments, where X is —NR¹⁴—NR¹⁵—, and R¹⁴ and R¹⁵ are hydrogen as shown below, the sum of m+n+o is 2, 3 or 4 and/or the sum of p+q+r is 1, 2, 3 or 4.

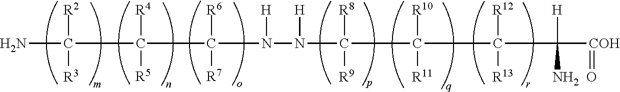

In certain embodiments, where X is —S—S—, as shown below, the sum of m+n+o is 2, 3 or 4 and/or the sum of p+q+r is 1, 2, 3 or 4.

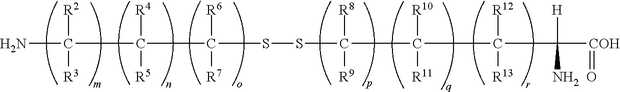

In some embodiments of the aforesaid embodiments defining sums of m+n+o and/or defining sums of p+q+r, each of R² through R¹³ is independently selected from hydrogen and —(C₁-C₈)alkyl. In certain embodiments, R² through R¹³ are hydrogen.

In certain embodiments, R¹ is selected from hydrogen and —(C₁-C₈)alkyl. In certain embodiments, R¹⁴ and R¹⁵ are independently selected from hydrogen or —(C₁-C₈)alkyl. In certain embodiments, R¹, R¹⁴, and R¹⁵ are independently selected from hydrogen and —(C₁-C₈)alkyl. In the aforementioned embodiments, the —(C₁-C₈)alkyl is preferably —(C₁-C₆)alkyl, more preferably —(C₁-C₃)alkyl, more preferably methyl or ethyl. In certain embodiments, R¹, R¹⁴, and R¹⁵ are hydrogen.

In certain embodiments, each of R², R³, R⁴, R⁵, R⁶, and R⁷ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R², R³, R⁴, R⁵, R⁶, and R⁷ are hydrogen.

In certain embodiments, each of R⁸, R⁹, R¹⁰, R¹¹, R¹², and R¹³ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R⁸, R⁹, R¹⁰, R¹¹, R¹², and R¹³ are hydrogen.

In certain embodiments, each of R² through R¹³ are independently selected from hydrogen and —(C₁-C₈)alkyl, according to the above schemes. In certain embodiments, R² through R¹³ are hydrogen.

In an embodiment, the compound of Formula I is (S)-2-amino-3-(3-aminopropoxy)propanoic acid.

Exemplary compounds of the Formula II are set forth in Table 1.

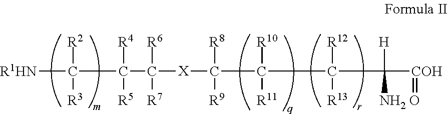

TABLE 1
Exemplary compounds of the Formula (H)

| R¹ | R² | R³ | R⁴ | R⁵ | R⁶ | R⁷ | R⁸ | R⁹ | R¹⁰ | R¹¹ | R¹² | R¹³ | m | q | r |
|---|---|---|---|---|---|---|---|---|---|---|---|---|---|---|---|
| H | H | H | H | H | H | H | H | H | NA | NA | NA | NA | 1 | 0 | 0 |
| H | H | H | H | H | H | H | H | H | NA | NA | NA | NA | 2 | 0 | 0 |
| H | H | H | H | H | H | H | H | H | NA | NA | NA | NA | 3 | 0 | 0 |
| H | H | H | H | H | H | H | H | H | NA | NA | NA | NA | 4 | 0 | 0 |
| H | H | H | H | H | H | H | H | H | NA | NA | H | H | 1 | 0 | 1 |
| H | H | H | H | H | H | H | H | H | NA | NA | H | H | 2 | 0 | 1 |
| H | H | H | H | H | H | H | H | H | NA | NA | H | H | 3 | 0 | 1 |
| H | H | H | H | H | H | H | H | H | NA | NA | H | H | 4 | 0 | 1 |
| H | NA | NA | H | H | H | H | H | H | H | H | H | H | 0 | 1 | 1 |
| H | H | H | H | H | H | H | H | H | H | H | H | H | 1 | 1 | 1 |
| H | H | H | H | H | H | H | H | H | H | H | H | H | 2 | 1 | 1 |
| H | H | H | H | H | H | H | H | H | H | H | H | H | 3 | 1 | 1 |
| H | H | H | H | H | H | H | H | H | H | H | H | H | 4 | 1 | 1 |
| H | H | H | H | H | H | H | H | H | H | H | H | H | 0 | 2 | 1 |
| H | H | H | H | H | H | H | H | H | H | H | H | H | 1 | 2 | 1 |
| H | H | H | H | H | H | H | H | H | H | H | H | H | 2 | 2 | 1 |
| Me | NA | NA | H | H | H | H | H | H | NA | NA | NA | NA | 0 | 0 | 0 |
| Me | H | H | H | H | H | H | H | H | NA | NA | NA | NA | 1 | 0 | 0 |
| Me | NA | NA | H | H | H | H | H | H | NA | NA | H | H | 0 | 0 | 1 |
| Me | H | H | H | H | H | H | H | H | NA | NA | NA | NA | 1 | 0 | 0 |
| H | H | H | Me | H | H | H | H | H | H | H | H | H | 1 | 1 | 1 |
| Me | H | H | OH | H | H | H | H | H | H | H | NA | NA | 1 | 1 | 0 |
| H | H | H | H | H | H | H | H | H | Me | H | H | H | 2 | 1 | 1 |
| iPr | H | H | H | H | H | H | H | H | H | OH | H | H | 3 | 1 | 1 |
| 2-Cl | H | H | H | Me | H | n-hexyl | H | H | H | H | H | i-Pr | 2 | 2 | 1 |
| benzyl | | | | | | | | | | | | | | | |
| Pyridin- | H | H | H | H | H | H | H | H | benzyl | H | H | H | 5 | 1 | 2 |
| 4-yl- | | | | | | | | | | | | | | | |
| methyl | | | | | | | | | | | | | | | |

Exemplary compounds of the Formula III are set forth in Table 2.

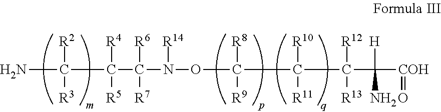

TABLE 2
Exemplary compounds of the Formula (III)

| R² | R³ | R⁴ | R⁵ | R⁶ | R⁷ | R⁸ | R⁹ | R¹⁰ | R¹¹ | R¹² | R¹³ | R¹⁴ | m | p | q |
|---|---|---|---|---|---|---|---|---|---|---|---|---|---|---|---|
| H | H | H | H | H | H | NA | NA | NA | NA | H | H | H | 1 | 0 | 0 |
| H | H | H | H | H | H | NA | NA | NA | NA | H | H | H | 2 | 0 | 0 |
| H | H | H | H | H | H | NA | NA | NA | NA | H | H | H | 3 | 0 | 0 |
| H | H | H | H | H | H | NA | NA | NA | NA | H | H | H | 4 | 0 | 0 |
| H | H | H | H | H | H | NA | NA | H | H | H | H | H | 1 | 0 | 1 |
| H | H | H | H | H | H | NA | NA | H | H | H | H | H | 2 | 0 | 1 |
| H | H | H | H | H | H | NA | NA | H | H | H | H | H | 3 | 0 | 1 |
| H | H | H | H | H | H | NA | NA | H | H | H | H | H | 4 | 0 | 1 |
| NA | NA | H | H | H | H | H | H | H | H | H | H | H | 0 | 1 | 1 |
| H | H | H | H | H | H | H | H | H | H | H | H | H | 1 | 1 | 1 |
| H | H | H | H | H | H | H | H | H | H | H | H | H | 2 | 1 | 1 |
| H | H | H | H | H | H | H | H | H | H | H | H | H | 3 | 1 | 1 |
| H | H | H | H | H | H | H | H | H | H | H | H | H | 4 | 1 | 1 |
| NA | NA | H | H | H | H | H | H | H | H | H | H | H | 0 | 2 | 1 |
| H | H | H | H | H | H | H | H | H | H | H | H | H | 1 | 2 | 1 |
| H | H | H | H | H | H | H | H | H | H | H | H | H | 2 | 2 | 1 |
| NA | NA | H | H | H | H | NA | NA | NA | NA | H | H | Me | 0 | 0 | 0 |
| H | H | H | H | H | H | NA | NA | NA | NA | H | H | Me | 1 | 0 | 0 |
| NA | NA | H | H | H | H | NA | NA | H | H | H | H | Me | 0 | 0 | 1 |
| H | H | H | H | H | H | NA | NA | NA | NA | H | H | Me | 1 | 0 | 0 |
| H | H | Me | H | H | H | H | H | H | H | H | H | H | 1 | 1 | 1 |
| H | H | OH | H | H | H | H | H | NA | NA | H | H | Me | 1 | 1 | 0 |
| H | H | H | H | H | H | H | H | Me | H | H | H | H | 2 | 1 | 1 |
| H | H | H | H | H | H | H | H | H | OH | H | H | iPr | 3 | 1 | 1 |
| H | H | H | Me | H | n-hexyl | H | H | H | H | H | i-Pr | 2-Cl | 2 | 2 | 1 |
| | | | | | | | | | | | | benzyl | | | |
| H | H | H | H | H | H | H | H | benzyl | H | H | H | Pyridin- | 5 | 1 | 2 |
| | | | | | | | | | | | | 4-yl- | | | |
| | | | | | | | | | | | | methyl | | | |

Exemplary compounds of the Formula IV are set forth in Table 3.

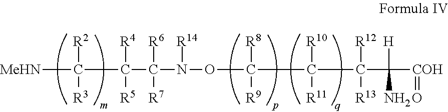

TABLE 3
Examplary compounds of the Formula (IV)

| R² | R³ | R⁴ | R⁵ | R⁶ | R⁷ | R⁸ | R⁹ | R¹⁰ | R¹¹ | R¹² | R¹³ | R¹⁴ | m | p | q |
|---|---|---|---|---|---|---|---|---|---|---|---|---|---|---|---|
| H | H | H | H | H | H | NA | NA | NA | NA | H | H | H | 1 | 0 | 0 |
| H | H | H | H | H | H | NA | NA | NA | NA | H | H | H | 2 | 0 | 0 |
| H | H | H | H | H | H | NA | NA | NA | NA | H | H | H | 3 | 0 | 0 |
| H | H | H | H | H | H | NA | NA | NA | NA | H | H | H | 4 | 0 | 0 |
| H | H | H | H | H | H | NA | NA | H | H | H | H | H | 1 | 0 | 1 |
| H | H | H | H | H | H | NA | NA | H | H | H | H | H | 2 | 0 | 1 |
| H | H | H | H | H | H | NA | NA | H | H | H | H | H | 3 | 0 | 1 |
| H | H | H | H | H | H | NA | NA | H | H | H | H | H | 4 | 0 | 1 |
| NA | NA | H | H | H | H | H | H | H | H | H | H | H | 0 | 1 | 1 |
| H | H | H | H | H | H | H | H | H | H | H | H | H | 1 | 1 | 1 |
| H | H | H | H | H | H | H | H | H | H | H | H | H | 2 | 1 | 1 |
| H | H | H | H | H | H | H | H | H | H | H | H | H | 3 | 1 | 1 |
| H | H | H | H | H | H | H | H | H | H | H | H | H | 4 | 1 | 1 |
| NA | NA | H | H | H | H | H | H | H | H | H | H | H | 0 | 2 | 1 |
| H | H | H | H | H | H | H | H | H | H | H | H | H | 1 | 2 | 1 |
| H | H | H | H | H | H | H | H | H | H | H | H | H | 2 | 2 | 1 |
| NA | NA | H | H | H | H | NA | NA | NA | NA | H | H | Me | 0 | 0 | 0 |
| H | H | H | H | H | H | NA | NA | NA | NA | H | H | Me | 1 | 0 | 0 |
| NA | NA | H | H | H | H | NA | NA | H | H | H | H | Me | 0 | 0 | 1 |
| H | H | H | H | H | H | NA | NA | NA | NA | H | H | Me | 1 | 0 | 0 |
| H | H | Me | H | H | H | H | H | H | H | H | H | H | 1 | 1 | 1 |
| H | H | OH | H | H | H | H | H | NA | NA | H | H | Me | 1 | 1 | 0 |
| H | H | H | H | H | H | H | H | Me | H | H | H | H | 2 | 1 | 1 |
| H | H | H | H | H | H | H | H | H | OH | H | H | iPr | 3 | 1 | 1 |
| H | H | H | Me | H | n-hexyl | H | H | H | H | H | i-Pr | 2-Cl | 2 | 2 | 1 |
| | | | | | | | | | | | | benzyl | | | |
| H | H | H | H | H | H | H | H | benzyl | H | H | H | Pyridin- | 5 | 1 | 2 |
| | | | | | | | | | | | | 4-yl- | | | |
| | | | | | | | | | | | | methyl | | | |

Exemplary compounds of the Formula V are set forth in Table 4.

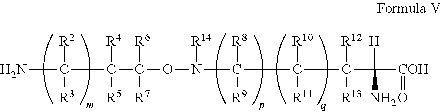

TABLE 4
Exemplary compounds of the Formula (V)

| R² | R³ | R⁴ | R⁵ | R⁶ | R⁷ | R⁸ | R⁹ | R¹⁰ | R¹¹ | R¹² | R¹³ | R¹⁴ | m | p | q |
|---|---|---|---|---|---|---|---|---|---|---|---|---|---|---|---|
| H | H | H | H | H | H | NA | NA | NA | NA | H | H | H | 1 | 0 | 0 |
| H | H | H | H | H | H | NA | NA | NA | NA | H | H | H | 2 | 0 | 0 |
| H | H | H | H | H | H | NA | NA | NA | NA | H | H | H | 3 | 0 | 0 |
| H | H | H | H | H | H | NA | NA | NA | NA | H | H | H | 4 | 0 | 0 |
| H | H | H | H | H | H | NA | NA | H | H | H | H | H | 1 | 0 | 1 |
| H | H | H | H | H | H | NA | NA | H | H | H | H | H | 2 | 0 | 1 |
| H | H | H | H | H | H | NA | NA | H | H | H | H | H | 3 | 0 | 1 |
| H | H | H | H | H | H | NA | NA | H | H | H | H | H | 4 | 0 | 1 |
| NA | NA | H | H | H | H | H | H | H | H | H | H | H | 0 | 1 | 1 |
| H | H | H | H | H | H | H | H | H | H | H | H | H | 1 | 1 | 1 |
| H | H | H | H | H | H | H | H | H | H | H | H | H | 2 | 1 | 1 |
| H | H | H | H | H | H | H | H | H | H | H | H | H | 3 | 1 | 1 |
| H | H | H | H | H | H | H | H | H | H | H | H | H | 4 | 1 | 1 |
| NA | NA | H | H | H | H | H | H | H | H | H | H | H | 0 | 2 | 1 |
| H | H | H | H | H | H | H | H | H | H | H | H | H | 1 | 2 | 1 |
| H | H | H | H | H | H | H | H | H | H | H | H | H | 2 | 2 | 1 |
| NA | NA | H | H | H | H | NA | NA | NA | NA | H | H | Me | 0 | 0 | 0 |
| H | H | H | H | H | H | NA | NA | NA | NA | H | H | Me | 1 | 0 | 0 |
| NA | NA | H | H | H | H | NA | NA | H | H | H | H | Me | 0 | 0 | 1 |
| H | H | H | H | H | H | NA | NA | NA | NA | H | H | Me | 1 | 0 | 0 |
| H | H | Me | H | H | H | H | H | H | H | H | H | H | 1 | 1 | 1 |
| H | H | OH | H | H | H | H | H | NA | NA | H | H | Me | 1 | 1 | 0 |
| H | H | H | H | H | H | H | H | Me | H | H | H | H | 2 | 1 | 1 |
| H | H | H | H | H | H | H | H | H | OH | H | H | iPr | 3 | 1 | 1 |
| H | H | H | Me | H | n-hexyl | H | H | H | H | H | i-Pr | 2-Cl | 2 | 2 | 1 |
| | | | | | | | | | | | | benzyl | | | |
| H | H | H | H | H | H | H | H | benzyl | H | H | H | Pyridin- | 5 | 1 | 2 |
| | | | | | | | | | | | | 4-yl- | | | |
| | | | | | | | | | | | | methyl | | | |

Exemplary compounds of the Formula VI are set forth in Table 5.

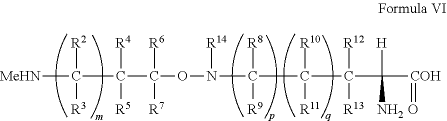

TABLE 5
Exemplary compounds of the Formula (VI)

| R² | R³ | R⁴ | R⁵ | R⁶ | R⁷ | R⁸ | R⁹ | R¹⁰ | R¹¹ | R¹² | R¹³ | R¹⁴ | m | p | q |
|---|---|---|---|---|---|---|---|---|---|---|---|---|---|---|---|
| H | H | H | H | H | H | NA | NA | NA | NA | H | H | H | 1 | 0 | 0 |
| H | H | H | H | H | H | NA | NA | NA | NA | H | H | H | 2 | 0 | 0 |
| H | H | H | H | H | H | NA | NA | NA | NA | H | H | H | 3 | 0 | 0 |
| H | H | H | H | H | H | NA | NA | NA | NA | H | H | H | 4 | 0 | 0 |
| H | H | H | H | H | H | NA | NA | H | H | H | H | H | 1 | 0 | 1 |
| H | H | H | H | H | H | NA | NA | H | H | H | H | H | 2 | 0 | 1 |
| H | H | H | H | H | H | NA | NA | H | H | H | H | H | 3 | 0 | 1 |
| H | H | H | H | H | H | NA | NA | H | H | H | H | H | 4 | 0 | 1 |
| NA | NA | H | H | H | H | H | H | H | H | H | H | H | 0 | 1 | 1 |
| H | H | H | H | H | H | H | H | H | H | H | H | H | 1 | 1 | 1 |
| H | H | H | H | H | H | H | H | H | H | H | H | H | 2 | 1 | 1 |
| H | H | H | H | H | H | H | H | H | H | H | H | H | 3 | 1 | 1 |
| H | H | H | H | H | H | H | H | H | H | H | H | H | 4 | 1 | 1 |
| NA | NA | H | H | H | H | H | H | H | H | H | H | H | 0 | 2 | 1 |
| H | H | H | H | H | H | H | H | H | H | H | H | H | 1 | 2 | 1 |
| H | H | H | H | H | H | H | H | H | H | H | H | H | 2 | 2 | 1 |
| NA | NA | H | H | H | H | NA | NA | NA | NA | H | H | Me | 0 | 0 | 0 |
| H | H | H | H | H | H | NA | NA | NA | NA | H | H | Me | 1 | 0 | 0 |
| NA | NA | H | H | H | H | NA | NA | H | H | H | H | Me | 0 | 0 | 1 |
| H | H | H | H | H | H | NA | NA | NA | NA | H | H | Me | 1 | 0 | 0 |
| H | H | Me | H | H | H | H | H | H | H | H | H | H | 1 | 1 | 1 |
| H | H | OH | H | H | H | H | H | NA | NA | H | H | Me | 1 | 1 | 0 |
| H | H | H | H | H | H | H | H | Me | H | H | H | H | 2 | 1 | 1 |
| H | H | H | H | H | H | H | H | H | OH | H | H | iPr | 3 | 1 | 1 |
| H | H | H | Me | H | n-hexyl | H | H | H | H | H | i-Pr | 2-Cl | 2 | 2 | 1 |
| | | | | | | | | | | | | benzyl | | | |
| H | H | H | H | H | H | H | H | benzyl | H | H | H | Pyridin- | 5 | 1 | 2 |
| | | | | | | | | | | | | 4-yl- | | | |
| | | | | | | | | | | | | methyl | | | |

Exemplary compounds of the Formula VII are set forth in Table 6.

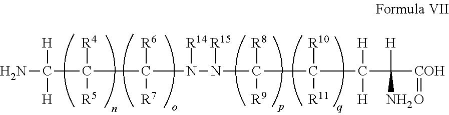

TABLE 6
Exemplary compounds of the Formula (VII)

| R⁴ | R⁵ | R⁶ | R⁷ | R⁸ | R⁹ | R¹⁰ | R¹¹ | R¹⁴ | R¹⁵ | n | o | p | q |
|---|---|---|---|---|---|---|---|---|---|---|---|---|---|
| H | H | NA | NA | NA | NA | H | H | H | H | 1 | 0 | 0 | 1 |
| H | H | NA | NA | NA | NA | H | H | Me | H | 1 | 0 | 0 | 1 |
| H | H | NA | NA | NA | NA | H | H | H | Me | 1 | 0 | 0 | 1 |
| H | H | NA | NA | NA | NA | H | H | Me | Me | 1 | 0 | 0 | 1 |
| H | H | NA | NA | H | H | H | H | H | H | 1 | 0 | 1 | 1 |
| H | H | NA | NA | H | H | H | H | Et | H | 1 | 0 | 1 | 1 |
| H | H | NA | NA | H | H | H | H | H | iPr | 1 | 0 | 1 | 1 |
| H | H | NA | NA | H | H | H | H | Me | benzyl | 1 | 0 | 1 | 1 |
| H | H | H | H | H | H | H | H | H | H | 1 | 1 | 2 | 1 |
| H | H | H | Me | H | H | H | Et | H | H | 1 | 1 | 2 | 1 |
| OH | H | H | H | H | H | 2-Cl | H | H | H | 1 | 1 | 2 | 1 |
| | | | | | | benzyl | | | | | | | |
| H | H | H | H | H | H | Et | H | H | H | 2 | 2 | 1 | 2 |
| H | H | H | Pyridin- | H | H | H | H | H | H | 1 | 1 | 1 | 2 |
| | | | 4-yl- | | | | | | | | | | |
| | | | methyl | | | | | | | | | | |
| H | H | H | H | H | H | 2-Cl | H | n-Pr | H | 1 | 1 | 2 | 1 |
| | | | | | | benzyl | | | | | | | |
| H | H | H | H | H | H | H | Pyridin- | H | Me | 1 | 2 | 1 | 1 |
| | | | | | | | 4-yl- | | | | | | |
| | | | | | | | methyl | | | | | | |
| H | H | H | H | H | H | H | H | Et | H | 2 | 2 | 1 | |
| OH | H | H | H | H | H | H | H | Me | benzyl | 1 | 1 | 1 | 1 |
| H | H | H | H | H | H | benzyl | H | Pyridin- | Pyridin- | 5 | 1 | 2 | |
| | | | | | | | | 4-yl- | 4-yl- | | | | |
| | | | | | | | | methyl | methyl | | | | |

Exemplary compounds of the Formula VIII are set forth in Table 7.

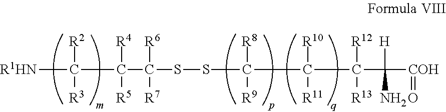

TABLE 7
Exemplary compounds of the Formula (VIII)

| R1 | R2 | R3 | R⁴ | R⁵ | R⁶ | R⁷ | R⁸ | R⁹ | R¹⁰ | R¹¹ | R¹² | R¹³ | m | p | q |
|---|---|---|---|---|---|---|---|---|---|---|---|---|---|---|---|
| H | NA | NA | H | H | H | H | NA | NA | NA | NA | H | H | 0 | 0 | 0 |
| Me | NA | NA | H | H | H | H | NA | NA | NA | NA | H | H | 0 | 0 | 0 |
| H | H | H | H | H | H | H | NA | NA | NA | NA | H | H | 1 | 0 | 0 |
| n- | H | H | H | H | H | H | NA | NA | NA | NA | H | H | 1 | 0 | 0 |
| Pr | | | | | | | | | | | | | | | |
| H | H | H | Me | H | H | H | H | H | H | H | H | H | 1 | 0 | 1 |
| Et | H | H | H | H | H | H | H | H | H | ben- | H | H | 1 | 0 | 1 |
| | | | | | | | | | | zyl | | | | | |
| H | H | H | OH | H | H | H | H | H | H | H | H | H | 3 | 1 | 2 |

Exemplary compounds of the Formula IX are set forth in Table 8.

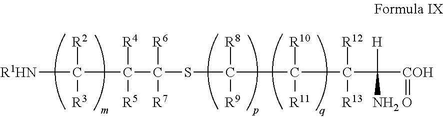

TABLE 8
Exemplary compounds of the Formula (IX)

| R1 | R2 | R3 | R⁴ | R⁵ | R⁶ | R⁷ | R⁸ | R⁹ | R¹⁰ | R¹¹ | R¹² | R¹³ | m | p | q |
|---|---|---|---|---|---|---|---|---|---|---|---|---|---|---|---|
| H | NA | NA | H | H | H | H | H | H | H | H | H | H | 0 | 1 | 1 |
| Me | NA | NA | H | H | H | H | H | H | H | H | H | H | 0 | 1 | 1 |
| H | NA | NA | H | H | H | H | H | H | Me | H | H | H | 0 | 1 | 1 |
| n- | NA | NA | Et | H | H | H | H | H | H | H | H | H | 0 | 1 | 1 |
| hexyl | | | | | | | | | | | | | | | |
| H | H | H | Me | H | H | H | H | H | H | H | H | H | 1 | 1 | 1 |
| ben- | H | H | H | H | H | H | H | H | H | H | H | H | 1 | 1 | 1 |
| zyl | | | | | | | | | | | | | | | |
| H | H | H | OH | H | H | H | NA | NA | NA | NA | H | H | 3 | 0 | 0 |
| Me | H | H | H | H | H | H | H | H | H | H | Me | Me | 4 | 0 | 0 |

The compounds having the structure of Formula I are for use in a method for increasing insulin sensitivity, reducing insulin resistance and/or preventing insulin resistance in a subject in need thereof. The method comprises to the subject an effective amount of a compound according to Formula I, or pharmaceutically acceptable salt thereof.

### Synthesis Schemes

Compounds of Formula I may be prepared according to Schemes 1-31.

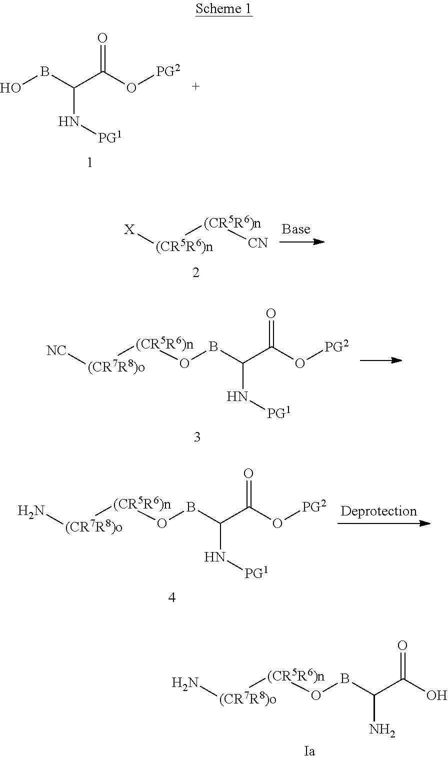

According to Scheme 1, a compound of the formula (I), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz) and PG² is selected, for example, from the group consisting of 9-fluorenylmethyl (Fm), C₁₋₆ alkyl and C₃₋₇ branched alkyl, is reacted with a compound of the formula (2), a known compound or compound prepared by known methods wherein X is a leaving group such as bromine, chlorine, iodine, methanesulfonate, tolylsulfonate, and the like, in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, or sodium t-butoxide, and the like, in a solvent such as tetrahydrofuran, 1,4-dioxane, methylene chloride, methanol, ethanol, t-butanol, and the like, optionally with heating, optionally with microwave irradiation, to provide a compound of the formula (3).

According to Scheme 1, a compound of the formula (3) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium, [1,1′-bis(diphenylphosphino) ferrocene]dichloropalladium, platinum on carbon, platinum on barium sulfate, platinum on celite, platinum on calcium carbonate, platinum on barium carbonate, platinum on silica, platinum on alumina, rhodium on carbon, rhodium on barium sulfate, rhodium on celite, rhodium on calcium carbonate, rhodium on barium carbonate, rhodium on silica, rhodium on alumina, and the like, in a solvent such as ethanol, methanol, tetrahydrofuran, 1,4-dioxane, ethyl acetate, benzene, toluene, cyclohexane, N,N-dimethylformamide, and the like, optionally with heating, optionally with microwave irradiation, to provide a compound of the formula (4).

According to Scheme 1, a compound of the formula (4) is then reacted with an acid such as acetic acid, trifluoroacetic acid, hydrochloric acid, sulfuric acid, trifluoromethanesulfonic acid, and the like, optionally in a solvent such as methylene chloride, tetrahydrofuran, 1,4-dioxane, ethanol, or methanol, and the like, optionally with heating, optionally with microwave irradiation, to provide a compound of the formula (Ia). Alternatively, a compound of the formula (4) is reacted with a base such as lithium hydroxide, sodium hydroxide, sodium carbonate, lithium carbonate, piperidine, pyridine, 2,6-lutidine, and the like, in a solvent such as methylene chloride, tetrahydrofuran, 1,4-dioxane, ethanol, or methanol, and the like, optionally with heating, optionally with microwave irradiation, to provide a compound of the formula (Ia). Alternatively, a compound of the formula (4) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, or methanol, and the like, optionally with heating, optionally with microwave irradiation, to provide a compound of the formula (Ia).

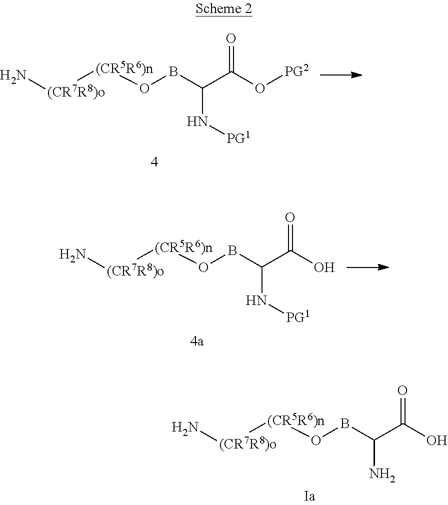

Alternatively, according to Scheme 2, a compound of the formula (4) is reacted with an acid such as acetic acid, trifluoroacetic acid, hydrochloric acid, sulfuric acid trifluoromethanesulfonic acid, and the like, optionally in a solvent such as methylene chloride, tetrahydrofuran, 1,4-dioxane, ethanol or methanol, and the like, optionally with heating, optionally with microwave irradiation to provide a compound of the formula (4a). Alternatively, according to Scheme 2, a compound of the formula (4) is reacted with a base such as lithium hydroxide, sodium hydroxide, sodium carbonate or lithium carbonate, and the like, in a solvent such as methylene chloride, tetrahydrofuran, 1,4-dioxane, ethanol or methanol, and the like, optionally with heating, optionally with microwave irradiation to provide a compound of the formula (4a).

According to Scheme 2, a compound of the formula (4a) is then reacted with a base such as piperidine, pyridine or 2,6-lutidine, and the like, in a solvent such as methylene chloride, tetrahydrofuran, 1,4-dioxane, ethanol or methanol, and the like, optionally with heating, optionally with microwave irradiation to provide a compound of the formula (Ia). Alternatively, a compound of the formula (4a) is reacted, according to Scheme 2, with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ia). Alternatively, a compound of the formula (4a) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (a).

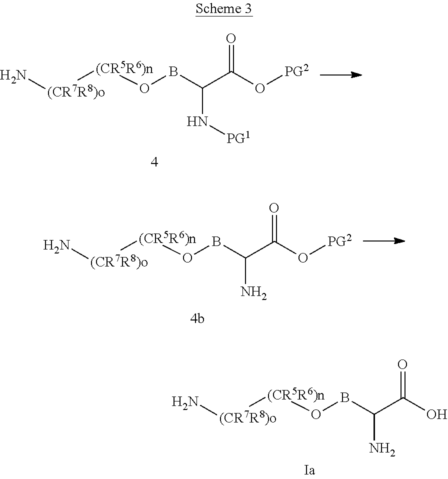

According to Scheme 3, a compound of the formula (4) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (4b). Alternatively, according to Scheme 3, a compound of the formula (4) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (4b).

According to Scheme 3, a compound of the formula (4b) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ia). Alternatively, according to Scheme 3, a compound of the formula (4b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, according to Scheme 3, a compound of the formula (4b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ia).

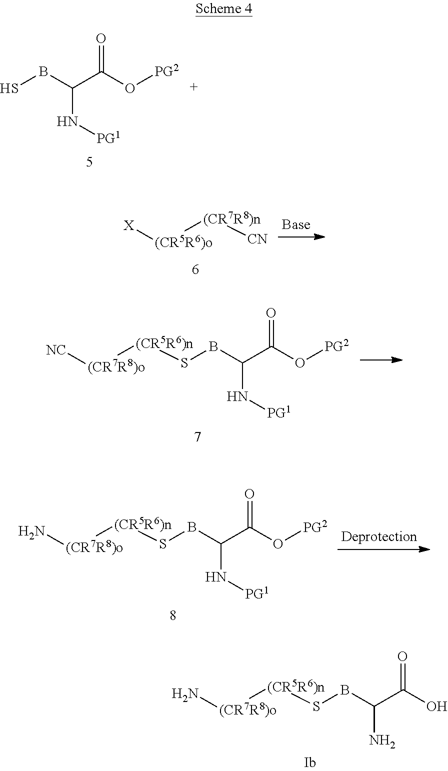

According to Scheme 4, a compound of the formula (5), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz) and PG is selected, for example, from the group consisting of 9-fluorenylmethyl (Fm), C₁₋₆ alkyl and C₃₋₇ branched alkyl, is reacted with a compound of the formula (6), a known compound or compound prepared by known methods wherein X is a leaving group such as bromine, chlorine, iodine, methanesulfonate, tolylsulfonate, and the like, in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, or sodium t-butoxide, and the like, in a solvent such as tetrahydrofuran, 1,4-dioxane, methylene chloride, methanol, ethanol, t-butanol, and the like, optionally with heating, optionally with microwave irradiation, to provide a compound of the formula (7).

According to Scheme 4, a compound of the formula (7) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium, [1,1′-bis(diphenylphosphino) ferrocene]dichloropalladium, platinum on carbon, platinum on barium sulfate, platinum on celite, platinum on calcium carbonate, platinum on barium carbonate, platinum on silica, platinum on alumina, rhodium on carbon, rhodium on barium sulfate, rhodium on celite, rhodium on calcium carbonate, rhodium on barium carbonate, rhodium on silica, rhodium on alumina, and the like, in a solvent such as ethanol, methanol, tetrahydrofuran, 1,4-dioxane, ethyl acetate, benzene, toluene, cyclohexane, N,N-dimethylformamide, and the like, optionally with heating, optionally with microwave irradiation, to provide a compound of the formula (8).

According to Scheme 4, a compound of the formula (8) is then reacted with an acid such as acetic acid, trifluoroacetic acid, hydrochloric acid, sulfuric acid, trifluoromethanesulfonic acid, and the like, optionally in a solvent such as methylene chloride, tetrahydrofuran, 1,4-dioxane, ethanol, or methanol, and the like, optionally with heating, optionally with microwave irradiation, to provide a compound of the formula (Ib). Alternatively, a compound of the formula (8) is reacted with a base such as lithium hydroxide, sodium hydroxide, sodium carbonate, lithium carbonate, piperidine, pyridine, 2,6-lutidine, and the like, in a solvent such as methylene chloride, tetrahydrofuran, 1,4-dioxane, ethanol, or methanol, and the like, optionally with heating, optionally with microwave irradiation, to provide a compound of the formula (Ib). Alternatively, a compound of the formula (8) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, or methanol, and the like, optionally with heating, optionally with microwave irradiation, to provide a compound of the formula (Ib).

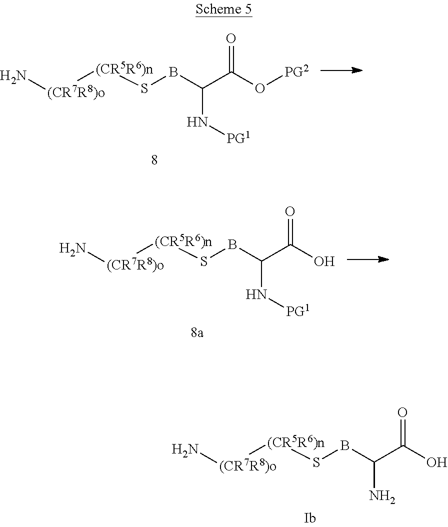

Alternatively, according to Scheme 5, a compound of the formula (8) is reacted with an acid such as acetic acid, trifluoroacetic acid, hydrochloric acid, sulfuric acid trifluoromethanesulfonic acid, and the like, optionally in a solvent such as methylene chloride, tetrahydrofuran, 1,4-dioxane, ethanol or methanol, and the like, optionally with heating, optionally with microwave irradiation to provide a compound of the formula (8a). Alternatively, according to Scheme 5, a compound of the formula (8) is reacted with a base such as lithium hydroxide, sodium hydroxide, sodium carbonate or lithium carbonate, and the like, in a solvent such as methylene chloride, tetrahydrofuran, 1,4-dioxane, ethanol or methanol, and the like, optionally with heating, optionally with microwave irradiation to provide a compound of the formula (8a).

According to Scheme 5, a compound of the formula (8a) is then reacted with a base such as piperidine, pyridine or 2,6-lutidine, and the like, in a solvent such as methylene chloride, tetrahydrofuran, 1,4-dioxane, ethanol or methanol, and the like, optionally with heating, optionally with microwave irradiation to provide a compound of the formula (Ib). Alternatively, a compound of the formula (8a) is reacted, according to Scheme 5, with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib). Alternatively, a compound of the formula (8a) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (Ib).

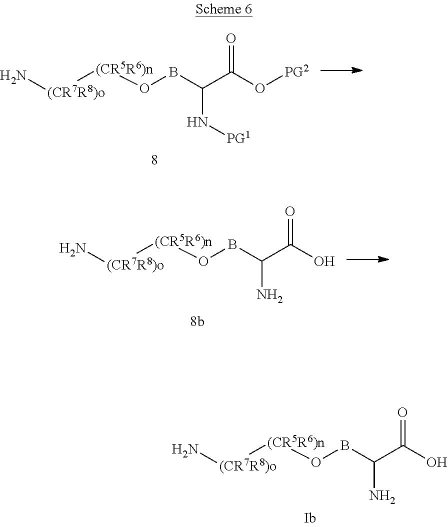

According to Scheme 6, a compound of the formula (8) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (8b). Alternatively, according to Scheme 6, a compound of the formula (8) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (8b).

According to Scheme 6, a compound of the formula (8b) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ib). Alternatively, according to Scheme 6, a compound of the formula (8b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib). Alternatively, according to Scheme 6, a compound of the formula (8b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ib).

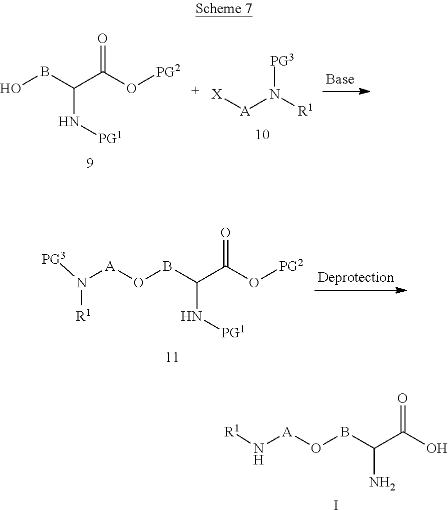

According to Scheme 7, a compound of the formula (9), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), and PG² is selected, for example, from the group consisting of 9-fluorenylmethyl (Fm), C₁₋₆ alkyl and C₃-7 branched alkyl, is reacted with a compound of the formula (10), a known compound or compound prepared by known methods wherein X is a leaving group such as bromine, chlorine, iodine, methanesulfonate, tolylsulfonate, and the like, and PG³ is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (11).

According to Scheme 7, a compound of the formula (11) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively a compound of the formula (11) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

Alternatively, according to Scheme 7, a compound of the formula (11) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I).

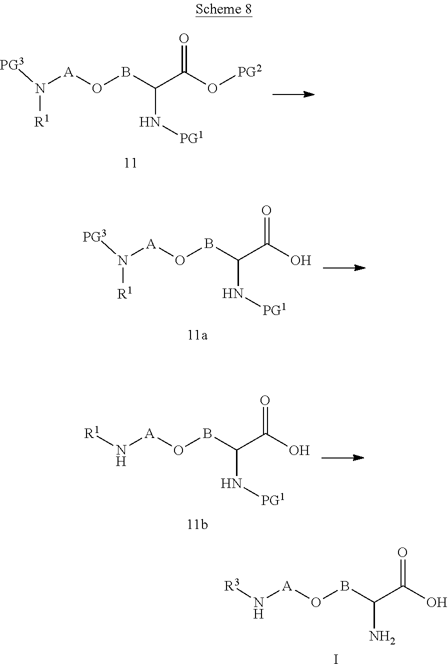

According to Scheme 8, a compound of the formula (11) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11a). Alternatively, a compound of the formula (11) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (11a).

According to Scheme 8, a compound of the formula (11a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (11b). Alternatively, a compound of the formula (11a) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (11b). Alternatively, a compound of the formula (11a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11b).

A compound of the formula (11b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (11b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, a compound of the formula (11b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

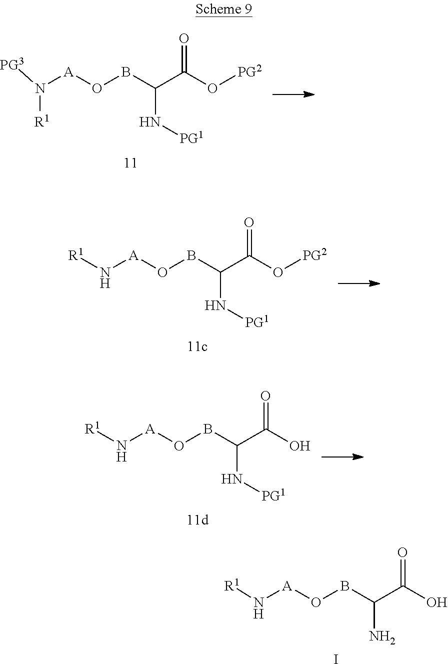

According to Scheme 9, a compound of the formula (11) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (11c). Alternatively, a compound of the formula (11) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (11c). Alternatively, a compound of the formula (11) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11c).

According to Scheme 9, a compound of the formula (11c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11d). Alternatively, a compound of the formula (11c) is reacted with a base such as lithium hydroxide, sodiumhydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (11d).

According to Scheme 9, a compound of the formula (11d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (11d) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (11d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

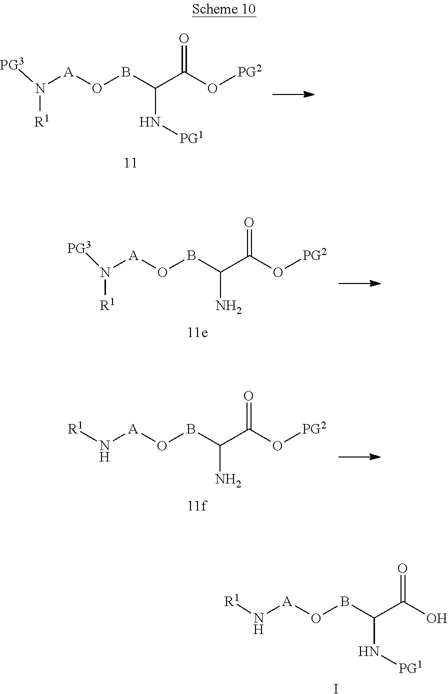

According to Scheme 10, a compound of the formula (11) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (11e). Alternatively, a compound of the formula (11) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (11e). Alternatively, a compound of the formula (11) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11e).

According to Scheme 10, a compound of the formula (111e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11f). Alternatively, a compound of the formula (11e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (11f).

According to Scheme 10, a compound of the formula (11f) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (11f) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (11f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

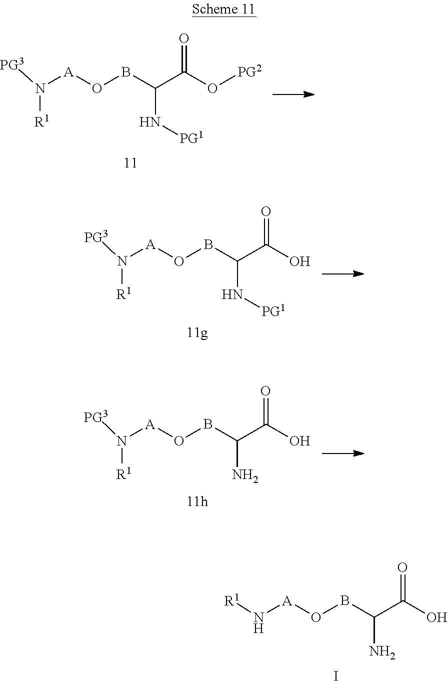

According to Scheme 11, a compound of the formula (11) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11g). Alternatively, a compound of the formula (11) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (11g).

According to Scheme 11, a compound of the formula (11g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (11h). Alternatively, a compound of the formula (11g) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (11h). Alternatively, a compound of the formula (11g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (11h).

According to Scheme 11, a compound of the formula (11h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (11h) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (11h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

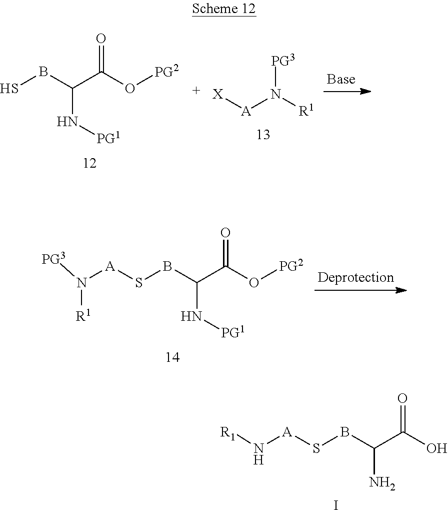

According to Scheme 12, a compound of the formula (12), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), and PG is selected, for example, from the group consisting of 9-fluorenylmethyl (Fm), C1-6 alkyl and C3-7 branched alkyl, is reacted with a compound of the formula (13), a known compound or compound prepared by known methods wherein X is a leaving group such as bromine, chlorine, iodine, methanesulfonate, tolylsulfonate, and the like, and PG³ is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (14).

According to Scheme 12, a compound of the formula (14) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively a compound of the formula (14) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

Alternatively, according to Scheme 12, a compound of the formula (14) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I).

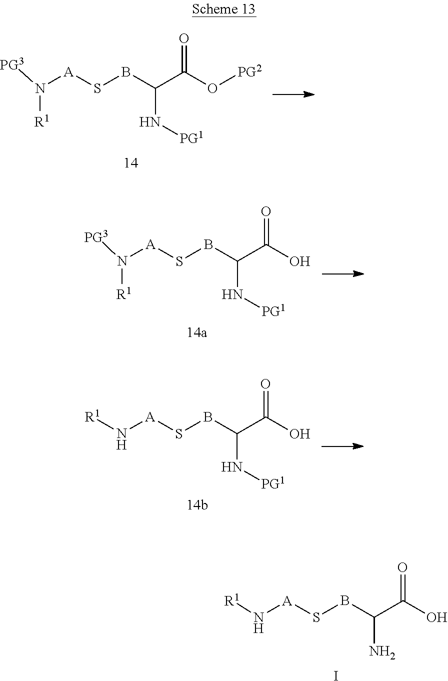

According to Scheme 13, a compound of the formula (14) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14a). Alternatively, a compound of the formula (14) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (14a).

According to Scheme 13, a compound of the formula (14a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (14b). Alternatively, a compound of the formula (14a) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (14b). Alternatively, a compound of the formula (14a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14b).

According to Scheme 13, compound of the formula (14b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (14b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, a compound of the formula (14b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

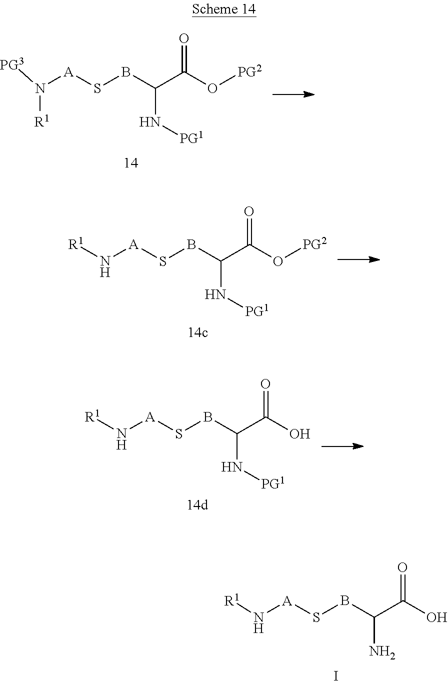

According to Scheme 14, a compound of the formula (14) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (14c). Alternatively, a compound of the formula (14) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (14c). Alternatively, a compound of the formula (14) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14c).

According to Scheme 14, a compound of the formula (14c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14d). Alternatively, a compound of the formula (14c) is reacted with a base such as lithium hydroxide, sodiumhydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (14d).

According to Scheme 14, a compound of the formula (14d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (14d) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (14d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

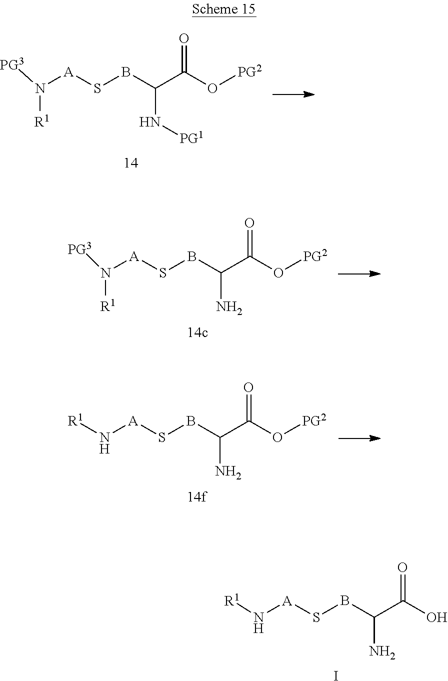

According to Scheme 15, a compound of the formula (14) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (14e). Alternatively, a compound of the formula (14) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (14e). Alternatively, a compound of the formula (14) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14e).

According to Scheme 15, a compound of the formula (14e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14f). Alternatively, a compound of the formula (14e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (14f).

According to Scheme 15, a compound of the formula (140 is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (14f) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (14f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

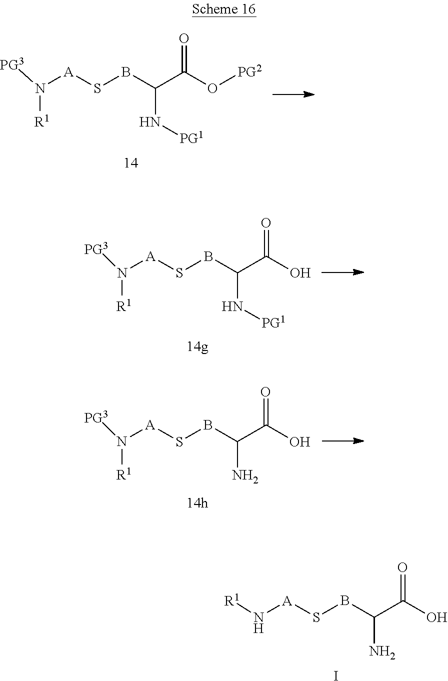

According to Scheme 16, a compound of the formula (14) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14g). Alternatively, a compound of the formula (14) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (14g).

According to Scheme 16, a compound of the formula (14g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (14h). Alternatively, a compound of the formula (14g) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (14h). Alternatively, a compound of the formula (14g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14h).

According to Scheme 16, a compound of the formula (14h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (14h) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (14h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

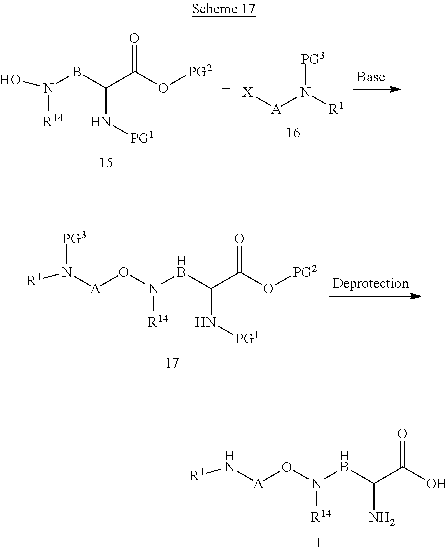

According to Scheme 17, a compound of the formula (15), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), and PG² is selected, for example, from the group consisting of 9-fluorenylmethyl (Fm), C1-6 alkyl and C3-7 branched alkyl, is reacted with a compound of the formula (16), a known compound or compound prepared by known methods wherein X is a leaving group such as bromine, chlorine, iodine, methanesulfonate, tolylsulfonate, and the like, and PG³ is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (17).

According to Scheme 17, a compound of the formula (17) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively a compound of the formula (17) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

Alternatively, according to Scheme 17, a compound of the formula (17) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium[1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I).

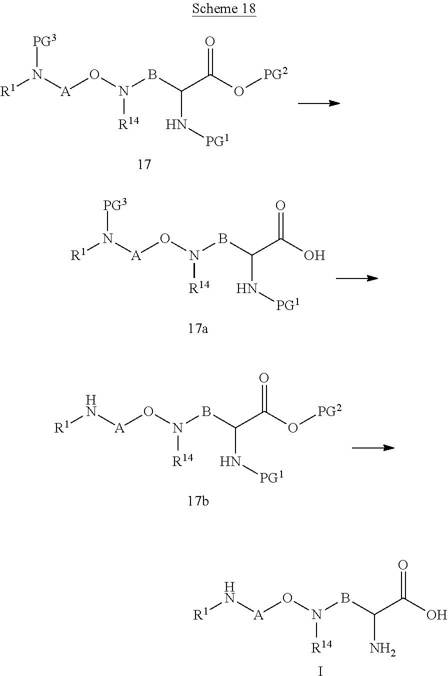

According to Scheme 18, a compound of the formula (17) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17a). Alternatively, a compound of the formula (17) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (17a).

According to Scheme 18, a compound of the formula (17a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (17b). Alternatively, a compound of the formula (17a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17b).

According to Scheme 18, a compound of the formula (17b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (17b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

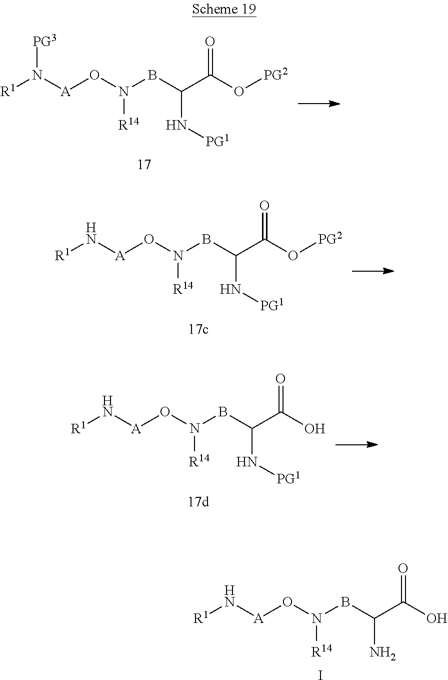

According to Scheme 19, a compound of the formula (17) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (17c). Alternatively, a compound of the formula (17) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17c).

According to Scheme 19, a compound of the formula (17c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17d). Alternatively, a compound of the formula (17c) is reacted with a base such as lithium hydroxide, sodiumhydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (17d).

According to Scheme 19, a compound of the formula (17d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (17d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

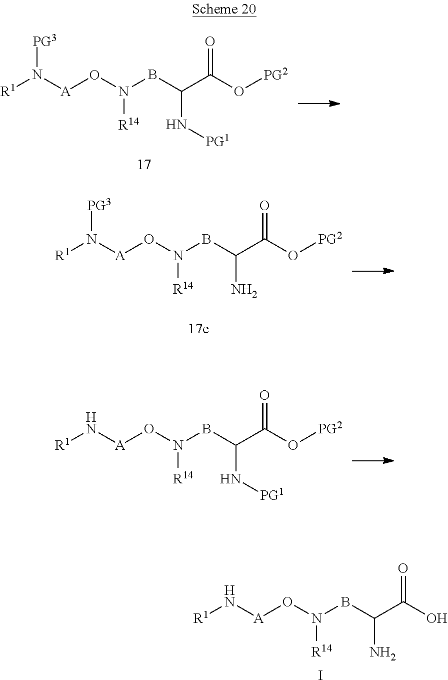

According to Scheme 20, a compound of the formula (17) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (17e). Alternatively, a compound of the formula (17) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17e).

According to Scheme 17, a compound of the formula (17e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (170. Alternatively, a compound of the formula (17e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (17f).

According to Scheme 20, a compound of the formula (17f) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (170 is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

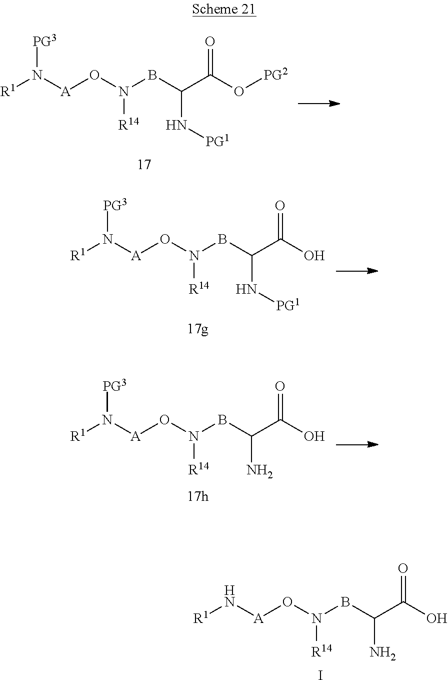

According to Scheme 21, a compound of the formula (17) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17g). Alternatively, a compound of the formula (17) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (17g).

According to Scheme 21, a compound of the formula (17g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (17h). Alternatively, a compound of the formula (17g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17h).

According to Scheme 21, a compound of the formula (17h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (17h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

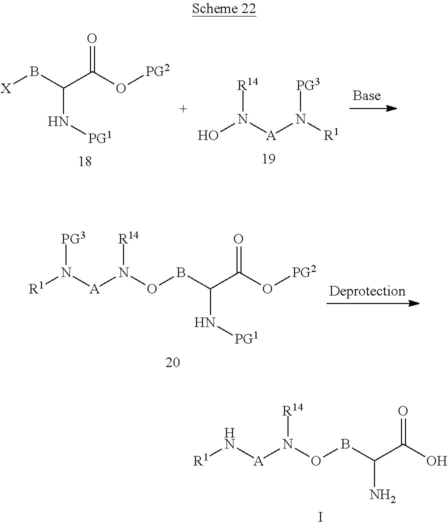

According to Scheme 22, a compound of the formula (18), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), PG is selected, for example, from the group consisting of 9-fluorenylmethyl (Fm), C1-6 alkyl and C3-7 branched alkyl and X is a leaving group such as bromine, chlorine, iodine, methanesulfonate, tolylsulfonate, and the like, is reacted with a compound of the formula (19), a known compound or compound prepared by known methods wherein PG3 is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (20).

According to Scheme 22, a compound of the formula (20) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively a compound of the formula (20) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

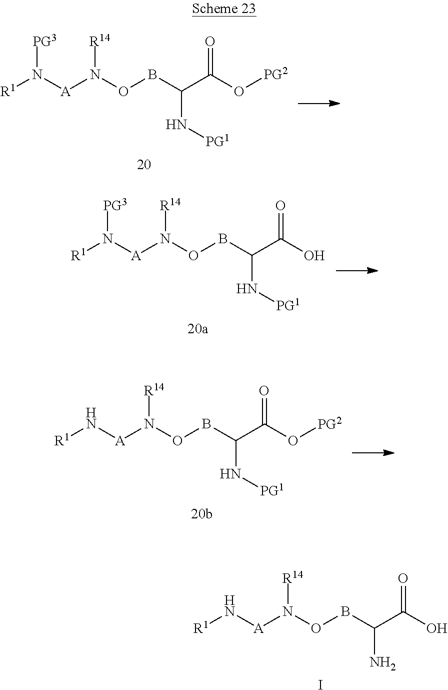

According to Scheme 22, a compound of the formula (20) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20a). Alternatively, a compound of the formula (20) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (20a).

According to Scheme 22, a compound of the formula (20a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (20b). Alternatively, a compound of the formula (20a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20b).

According to Scheme 22, a compound of the formula (20b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (20b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

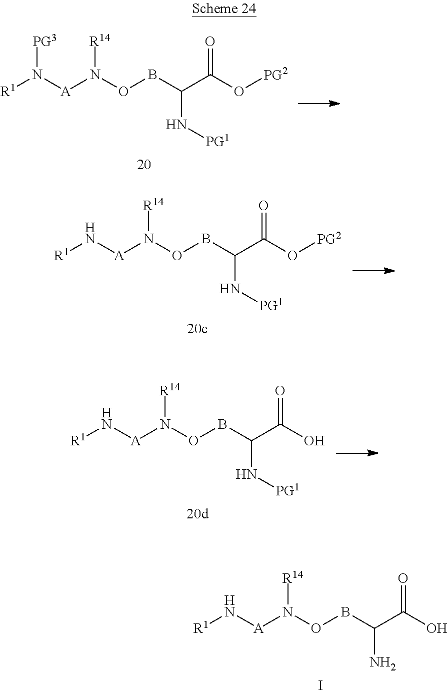

According to Scheme 24, a compound of the formula (20) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (20c). Alternatively, a compound of the formula (20) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20c).

According to Scheme 24, a compound of the formula (20c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20d). Alternatively, a compound of the formula (20c) is reacted with a base such as lithium hydroxide, sodiumhydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (20d).

According to Scheme 24, a compound of the formula (20d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (20d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

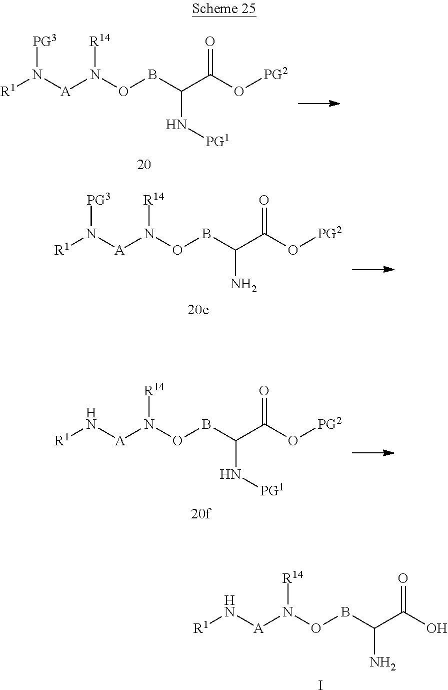

According to Scheme 25, a compound of the formula (29) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (20e). Alternatively, a compound of the formula (20) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20e).

According to Scheme 25, a compound of the formula (20e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20f). Alternatively, a compound of the formula (20e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (20f).

According to Scheme 25, a compound of the formula (20f) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (20f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

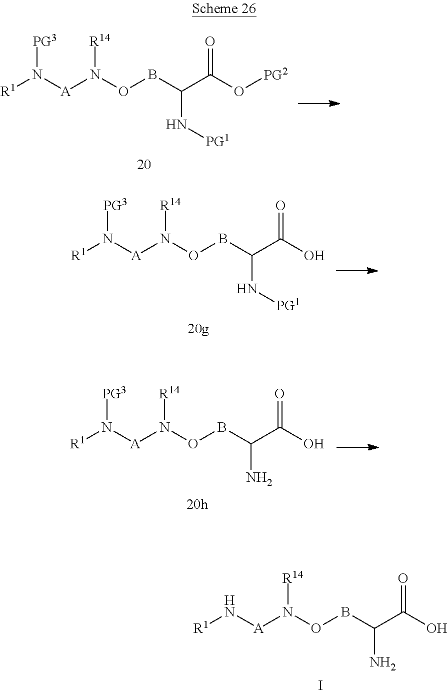

According to Scheme 26, a compound of the formula (20) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20g). Alternatively, a compound of the formula (20) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (20g).

According to Scheme 26, a compound of the formula (20g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (20h). Alternatively, a compound of the formula (20g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (20h).

According to Scheme 26, a compound of the formula (20h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (20h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

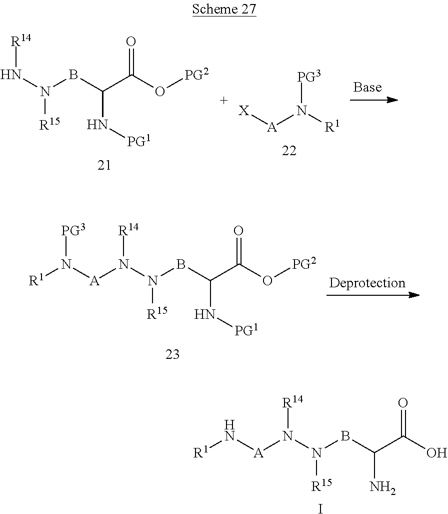

According to Scheme 27, a compound of the formula (21), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), and PG² is selected, for example, from the group consisting of 9-fluorenylmethyl (Fm), C1-6 alkyl and C3-7 branched alkyl, is reacted with a compound of the formula (22), a known compound or compound prepared by known methods wherein X is a leaving group such as bromine, chlorine, iodine, methanesulfonate, tolylsulfonate, and the like, and PG³ is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (23).

According to Scheme 27, a compound of the formula (23) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively a compound of the formula (23) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

Alternatively, according to Scheme 27, a compound of the formula (23) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I).

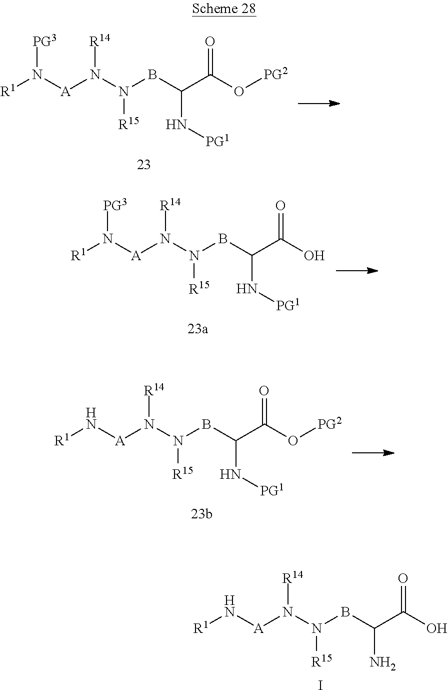

According to Scheme 28, a compound of the formula (23) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23a). Alternatively, a compound of the formula (23) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (23a).

According to Scheme 28, a compound of the formula (23a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (23b). Alternatively, a compound of the formula (23a) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (23b). Alternatively, a compound of the formula (23a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23b).

According to Scheme 28, a compound of the formula (23b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (23b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, a compound of the formula (23b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

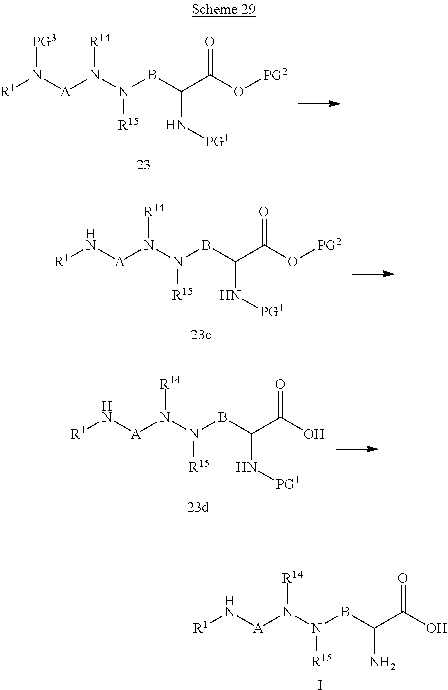

According to Scheme 29, a compound of the formula (23) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (23c). Alternatively, a compound of the formula (23) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (23c). Alternatively, a compound of the formula (23) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23c).

According to Scheme 29, a compound of the formula (23c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23d). Alternatively, a compound of the formula (23c) is reacted with a base such as lithium hydroxide, sodiumhydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (23d).

According to Scheme 29, a compound of the formula (23d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (23d) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (23d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

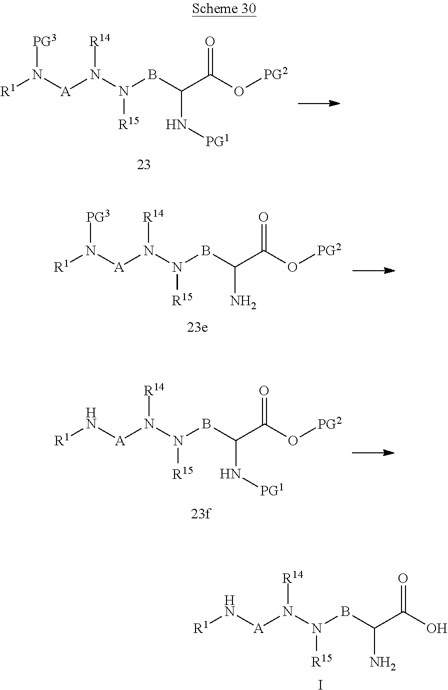

According to Scheme 30, a compound of the formula (23) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (23e). Alternatively, a compound of the formula (23) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (23e). Alternatively, a compound of the formula (23) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23e).

According to Scheme 30, a compound of the formula (23e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23f). Alternatively, a compound of the formula (23e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (23f).

According to Scheme 30, a compound of the formula (23f) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (23f) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (23f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

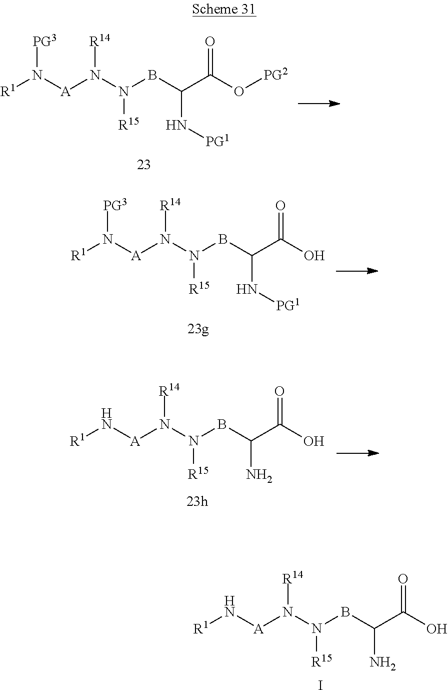

According to Scheme 31, a compound of the formula (23) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23g). Alternatively, a compound of the formula (23) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (23g).

According to Scheme 31, a compound of the formula (23g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (23h). Alternatively, a compound of the formula (23g) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (23h). Alternatively, a compound of the formula (23g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (23h).

According to Scheme 31, a compound of the formula (23h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (23h) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (23h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

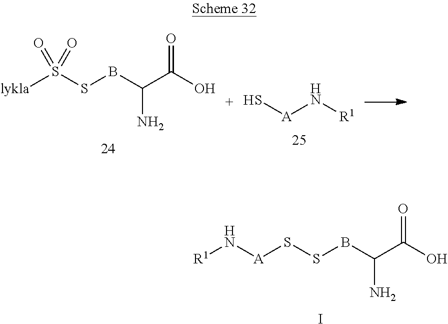

According to Scheme 32, a compound of the formula (24), a known compound or a compound prepared by known means wherein alkyl is an alkyl group selected, for example, from the group consisting of methyl, ethyl, propyl and isopropyl, is reacted with is reacted with a compound of the formula (25) in a solvent such as water, at an appropriate pH such as pH=4.0-6.0, optionally with heating, optionally with microwave irradiation to provide a compound of the formula (I).

In an embodiment, the compound of Formula I is (S)-2-amino-3-(3-aminopropoxy)propanoic acid:

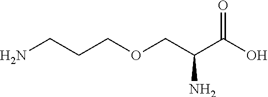 [SMILES: NCCCOC[C@H](N)C(=O)O]

A preferred salt is (S)-2-amino-3-(3-aminopropoxy)propanoic acid dihydrochloride:

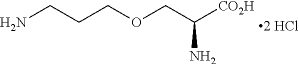 [SMILES: NCCCOC[C@H](N)C(=O)O]

An exemplary but non-limiting method of preparing (S)-2-amino-3-(3-aminopropoxy)propanoic acid dihydrochloride follows.

### (A) Preparation of (S)-methyl 2-((tert-butoxycarbonyl)amino)-3-(3-((tert-butoxycarbonyl)-amino)propoxy)propanoate

Sodium hydride (60% in mineral oil, 1.05 equivalents) is added to a stirred solution of (S)-methyl 2-((tert-butoxycarbonyl)amino)-3-hydroxypropanoate (1 eq.) in dry tetrahydrofuran under a nitrogen atmosphere with cooling in an ice/water bath. The resulting mixture is stirred with cooling for 1 hour and then a solution of tert-butyl (3-bromopropyl)carbamate (1.1 equivalent) in dry tetrahydrofuran is added dropwise over 10 minutes. The resulting reaction mixture is allowed to warm up to room temperature and then stirred overnight. The reaction is concentrated on a rotary evaporator and partitioned between ethyl acetate and water. The organic layer is separated, ished with brine, dried over anhydrous sodium sulfate and concentrated on a rotary evaporator to obtain a mixture that is purified by chromatography on silica gel (ethyl acetate/hexanes) to provide the desired product.

### (B) Preparation of (S)-2-((tert-butoxycarbonyl(amino)-3-(3-((tert-butoxycarbonyl)amino)-propoxy)propanoic acid

To a solution of (S)-methyl 2-((tert-butoxycarbonyl)amino)-3-(3-((tert-butoxycarbonyl)-amino)propoxy)propanoate in tetrahydrofuran is added an equal portion (v/v) of methanol, followed by a solution of lithium hydroxide hydrate dissolved in an equal portion (v/v) of water. The reaction is stirred at room temperature overnight. The resulting mixture is concentrated on a rotary evaporator, acidified with aqueous hydrochloric acid to a pH of 3-4 and extracted with ethyl acetate. The combined organic layers are dried over anhydrous magnesium sulfate and concentrated to afford the desired product.

### (C) Preparation of (S)-2-amino-3-(3-aminopropoxy)propanoic acid dihydrochloride

A solution of (S)-2-((tert-butoxycarbonyl(amino)-3-(3-((tert-butoxycarbonyl)amino)-propoxy)propanoic acid in 6N aqueous hydrochloric acid is refluxed for two hours. The resulting mixture is concentrated on a rotary evaporator to afford the desired product.

### Salts

The compounds of Formula I may take the form of salts when appropriately substituted with groups or atoms capable of forming salts. Such groups and atoms are well known to those of ordinary skill in the art of organic chemistry. The term “salts” embraces addition salts of free acids or free bases which are compounds of the invention. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, pivalic, propionic, furoic, mucic, isethionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, camphorosulfonic, and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, tromethamine, meglumine (N-methylglucamine) and procaine. Examples of pharmaceutically unacceptable base addition salts include lithium salts and cyanate salts.

All of these salts may be prepared by conventional means from the corresponding compound according to Formula I by reacting, for example, the appropriate acid or base with the compound according to Formula I. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selection, and Use by P. H. Stahl and C. G. Wermuth (Wiley-VCH 2002).

### Pharmaceutical Compositions and Therapeutic Administration

A pharmaceutical composition comprises a pharmaceutically acceptable carrier and a compound, or a pharmaceutically acceptable salt thereof, according to Formula I.

The compounds may be administered in the form of a pharmaceutical composition, in combination with a pharmaceutically acceptable carrier. The active ingredient or agent in such formulations (i.e., a compound of Formula I) may comprise from 0.1 to 99.99 weight percent of the formulation. “Pharmaceutically acceptable carrier” means any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,733,566 describes the use of polymeric microparticles that release antiparasitic compositions.

The controlled-release of the active ingredient may be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The compounds of Formula I may be administered in a convenient manner. Suitable topical routes include oral, rectal, inhaled (including nasal), topical (including buccal and sublingual), transdermal and vaginal, preferably across the epidermis. The compound of Formula I can also be used for parenteral administration (including subcutaneous, intravenous, intramuscular, intradermal, intraarterial, intrathecal and epidural), and the like. It will be appreciated that the preferred route may vary with for example the condition of the recipient.

A physician will determine the dosage of the active agent which will be most suitable and it will vary with the form of administration and the particular compound chosen, and furthermore, it will vary depending upon various factors, including but not limited to the patient under treatment and the age of the patient, the severity of the condition being treated, the rout of administration, and the like. A physician will generally wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 1000 mg, more typically from about 1 to about 500 mg, more typically, from about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The treatment may be carried out for as long a period as necessary, either in a single, uninterrupted session, or in discrete sessions. The treating physician will know how to increase, decrease, or interrupt treatment based on patient response. The treatment schedule may be repeated as required. According to one embodiment, compound of Formula I is administered once daily.

Treatment efficacy is generally determined by improvement in insulin resistance, i.e., an increase in insulin sensitivity. Insulin resistance may be assessed before, during and after treatment by use of the homeostasis model assessment of insulin resistance (HOMA-IR) index. The HOMA-IR value is calculated as level of fasting glucose (millimoles/liter) times the level of fasting insulin (microunits/milliliter) divided by 22.5. The value of 3.0 identifies the highest quartile among populations without diabetes (Ascaso et al., Diabetes Care, 2003, 26: 3320).

Treatment efficacy may also be assessed by the A1C test, which indicates the average of an individual's blood glucose level over the prior 3 months. See, Nathan, Diabetes Care, 32(12):e160 (2009).

The practice of the disclosed subject matter is illustrated by the following non-limiting examples.

### EXAMPLES

### Example 1

The following study demonstrates a direct link between function impairment due to GLUT4 carbonylation and insulin resistance. A GLUT4-SNAP fusion construct is generated. 3T3-L1 adipocytes were then retrovirally transduced with the construct to overexpress the GLUT4-SNAP protein. Twenty-four hours after the transduction, the cells were treated with and without 20 μM of 4-HNE for an additional 4 hours. This 4-HNE dose is chosen because it is similar to the physiological levels and is non-toxic. In order to identify and quantitate GLUT4 carbonylation (K264 NHE-adduct or R265 and R246 glutamic semialdehyde adducts), a mass spectroscopy-based multiple reaction monitoring (MRM) method is developed and validated. This high throughput method does not require antibodies, is robust and is sensitive at sub-picomole levels.

The results are shown in FIG. 1A-B, indicating that 4-HNE, 20 μM for 4 hour induced the formation of the K264-HNE adduct in 3T3-L1 cells overexpressing GLUT4. The MRM data presented in FIG. 1A shows an increase in the transition of HNE-induced K264-HNE adducts. The data is then used to calculate the amounts of carbonylated GLUT4 presented in FIG. 1B. Fourier transition of the GLUT4 peptide found in humans were used for quantitation.

### Example 2

The following study demonstrates the effect on of 4-HNE and H₂O₂ on glucose transport. 3T3-L adipocytes (1×10⁶) were treated with either 500 μM of 4-HNE or H₂O₂ or combination of both for 4 hour and were then stimulated with 100 nM insulin for 60 minutes. The glucose uptake is measured by a specific MRM method. The data, presented in FIG. 2, shows that glucose uptake by 3T3-11 adipocytes is reduced by 32% and 66% with 4-HNE and H₂O₂ treatment, respectively.

### Example 3

The following study demonstrates that GLUT4 carbonylation (adduction by 4HNE) is present in the fat tissue of insulin resistant, pre-diabetic and diabetic human individuals.

### A. Fat Tissue Sample Preparation

Proteins were extracted from the fat tissue (˜200 mg) using a Mem-PER Plus Membrane Protein Extraction Kit (Thermo Scientific #89842) and 1× protease inhibitor (Pierce Halt Protease Inhibitor Cocktail 100×). Tissue is incubated on ice for 10 min, then the homogenized for 2 min at 1000 rpm and proteins were separated by centrifugation at 10,000 g for 15 min.

### B. Digestion

Thirty microliters of each of the samples were denatured with 10 μl of DL-dithiothreitol (5 mg/ml) at 37° C. for 20 min and alkylated with 10 μL of iodoacetamide (12.5 mg/ml) at 37° C. for 20 min. Samples were diluted with 25 mM NH₄HCO₃ (450 μl) and 10 μL of trypsin is added for protein digestion. Formic acid 1% final concentration is used to stop the digestion and ionize the peptides.

### C. STAGE TIPS Treatment

Digested peptides were desalted and cleaned up by STAGE TIPS (Thermo Scientific, West Palm Beach, Fla.) using chromatographic C18 beads for immobilization. In brief, a solid phase C18 column is activated with acetonitrile 100% and equilibrated on buffer A (water −0.1% formic acid) the samples were passed two times through the tips, samples were desalted by ishing twice with buffer A and eluted with 50 μL of elution buffer (85% acetonitrile, 0.1% formic acid) and 50 μL of acetonitrile. Samples were dried in speed vacuum for 30 minutes and resuspended in the MRM analysis with 50 μl of 85% acetonitrile, 15% formic acid 0.1% and 115 μL of buffer A.

### D. Multiple Reaction Monitoring (MRM)

MRM-MS is a quantitative mass spectrometry-based target technology that generates unique fragment ions associated with their corresponding precursor ions. These ions can be detected and quantified in complex matrix samples. The quantitation of peptides is obtained measuring the intensity of the fragment ions. To detect carbonylated GLUT4, specific peptides were selected. The parent ion for the carbonylated GLUT4 sequence LTGWADVSGVLAELKDEK-4HNE (SEQ ID NO:2) with triple charge mass is 696.381 m/z.

After fragmentation of the parent ion, relative daughter ions: 1101.94, 788.4758, 917.5138, 988.5555, 722.4186, and 481.9482 m/z were identified and used for the quantification of the carbonylated Glut 4 peptide. Also, MRM methods for the GLUT4 unmodified sequence of the same peptide were also developed. Relative carbonylated GLUT4 is estimated as a percentage of the carbonylated peptide respect to the total of all GLUT4 peptide signal. An independent peptide from the heat shock 70 kDa protein 1A/1B (a protein not related to GLUT4) is selected as an internal standard. This internal standard peptide allows a relative quantification because it is used for normalization of the intensity of each GLUT4 peptide within the same sample.

MRM analyses were performed with a DIONEX UltiMate™ 3000 SRLCnano HPLC system (Thermo Scientific) coupled to a TSQ QUANTUM™ ULTRA (Thermo Scientific) triple quadrupole. PINPOINT™ software is used for method development and the optimization of the mass spectrometer parameters, as well as for peptide quantification. Peptides were separated by liquid chromatography, 5 μL of samples were injected in the nanoHPLC system and separation is carried out on a C18 column (ACCLAIM PepMap® RSLC, Thermo Scientific). Elution is performed with a gradient of at a flow rate of 0.300 μl/min with buffer A (0.1% (v/v) formic acid) and Buffer B (acetonitrile containing 0.1% (v/v) formic acid). A linear gradient is performed from 5% B to 30% B in 22.5 min followed by the ishing step with 90% B for 7 min and the column re-equilibration for 14 min, with a 50 min long total cycle. The mass spectroscopy analysis is carried out in a positive ionization mode, using an ion spray voltage of 2,000V and a temperature of 200 C. Nebulizer and gas flow is set at 30 psi.

The aforementioned MRM protocol is utilized to compare GLUT4 modifications in fat samples from the following groups: (i) lean insulin resistant; (ii) lean insulin resistant on overnutrition; (iii) obese non-diabetic; (iv) obese pre-diabetic and (v) obese diabetic. Comparing the adipose tissue of lean and lean-on-overnutrition insulin resistant individuals revealed that the levels of GLUT4-K264-NHE-adduct were increased by at-least 2.5 folds in subjects that were lean but insulin resistant and on a hypercaloric diet (FIG. 3A). Comparing the adipose tissue GLUT4 K264 NHE-adduct level in obese non-diabetic, obese pre-diabetic, and obese diabetic subjects revealed that the GLUT4 carbonylation is increased in the pre-diabetic and diabetic individuals (FIG. 3B).

To determine the percentage of GLUT4 that is carbonylated in the 5 study groups, an MRM method is employed to detect total GLUT4. The results in FIG. 3C show that increased levels of GLUT4 carbonylations occur in subjects that are insulin resistant, i.e., pre-diabetic and diabetic. About 50% GLUT4 carbonylation is identical to the reported˜50% decrease in insulin-stimulated glucose uptake (GIR) after 7 days of overnutrition.

The results in FIG. 3D confirm that the GLUT4 carbonylations linearly increase with the insulin resistance marker HbA1C (glycated haemoglobin) marker for insulin resistance.

### Example 4

### (S)-2-amino-3-(3-aminopropoxy)propanoic acid dihydrochloride

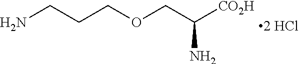 [SMILES: NCCCOC[C@H](N)C(=O)O]

(S)-2-amino-3-(3-aminopropoxy)propanoic acid dihydrochloride can be prepared as follows:

### (A) Preparation of (S)-methyl 2-((tert-butoxycarbonyl)amino)-3-(3-((tert-butoxycarbonyl)-amino)propoxy)propanoate

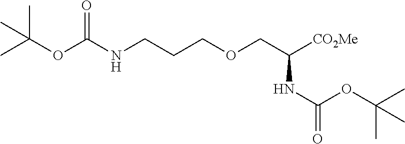 [SMILES: COC(=O)[C@H](COCCCNC(=O)OC(C)(C)C)NC(=O)OC(C)(C)C]

Sodium hydride (60% in mineral oil, 1.05 equivalents) is added to a stirred solution of (S)-methyl 2-((tert-butoxycarbonyl)amino)-3-hydroxypropanoate (1 eq.) in dry tetrahydrofuran under a nitrogen atmosphere with cooling in an ice/water bath. The resulting mixture is stirred with cooling for 1 hour and then a solution of tert-butyl (3-bromopropyl)carbamate (1.1 equivalent) in dry tetrahydrofuran is added dropwise over 10 minutes. The resulting reaction mixture is allowed to warm up to room temperature and then stirred overnight. The reaction is concentrated on a rotary evaporator and partitioned between ethyl acetate and water. The organic layer is separated, ished with brine, dried over anhydrous sodium sulfate and concentrated on a rotary evaporator to obtain a mixture that is purified by chromatography on silica gel (ethyl acetate/hexanes) to provide the desired product.

### (B) Preparation of (S)-2-((tert-butoxycarbonyl(amino)-3-(3-((tert-butoxycarbonyl)amino)-propoxy)propanoic acid

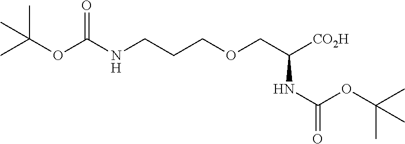 [SMILES: CC(C)(C)OC(=O)NCCCOC[C@H](NC(=O)OC(C)(C)C)C(=O)O]

To a solution of (S)-methyl 2-((tert-butoxycarbonyl)amino)-3-(3-((tert-butoxycarbonyl)-amino)propoxy)propanoate in tetrahydrofuran is added an equal portion (v/v) of methanol, followed by a solution of lithium hydroxide hydrate dissolved in an equal portion (v/v) of water. The reaction is stirred at room temperature overnight. The resulting mixture is concentrated on a rotary evaporator, acidified with aqueous hydrochloric acid to a pH of 3-4 and extracted with ethyl acetate. The combined organic layers are dried over anhydrous magnesium sulfate and concentrated to afford the desired product.

### (C) Preparation of (S)-2-amino-3-(3-aminopropoxy)propanoic acid dihydrochloride

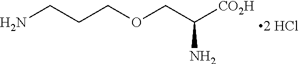 [SMILES: NCCCOC[C@H](N)C(=O)O]

A solution of (S)-2-((tert-butoxycarbonyl(amino)-3-(3-((tert-butoxycarbonyl)amino)-propoxy)propanoic acid in 6N aqueous hydrochloric acid is refluxed for two hours. The resulting mixture is concentrated on a rotary evaporator to afford the desired product.

Compounds of Formula I may be characterized by methods known in the art. The following are exemplary methods.

### Example 5

The following studies can be performed to demonstrate the solubility and solution stability of a Formula I compound.

### A. Solubility

The compound may be determined to be soluble in water or DMSO at a concentration of at least 10 mM. In phosphate buffered saline (PBS) solubility assay (PBS: 136.9 mM NaCl, 2.68 mM KCl, 8.1 mM Na₂HPO₄; 1.47 mM KH₂PO₄; +/−0.9 mM CaCl₂); +/−0.49 mM MgCl₂; pH 7.4), without Ca or Mg (Ca/Mg interfere with the assay), the compound may be found to have a solubility >200 μM (maximum concentration tested in a standard protocol).

### B. Liver Microsome Stability

The stability of the compound in the presence of liver microsomes is tested at 1 μM compound and 0.5 mg/ml rat or human microsomal protein, at 37° C. with 2 mM NADPH (standard in vitro screening concentrations). The “t_{1/2}” values are the maximum time employed for the respective assays. A preferred result is that no significant loss of the compound is seen at any time point in these assays.

### C. Solution Stability

The stability of the compound is tested in the following media, in the following concentrations: (i) 1 μM in mouse (male C57BL/6) plasma, (ii) 1 μM in PBS (with Ca and Mg) (control treatment); (iii) 5 μM in stimulated gastric fluid (SGF; 0.2% NaCl; 84 mM HCl; 0.32% pepsin; pH 1.2); (iv) 5 μM in stimulated intestinal fluid (SIF; 50 mM KP, pH 6.8; 10 mg/m pancreatin); and (v) 5 μM in PBS (+Ca and Mg) (control treatment). The “tin” values are the maximum time employed for the respective assays. A preferred result is that no significant loss of the compound is seen at any time point in these assays.

### Example 6

### Intravenous and Oral Pharmacokinetic Studies

The following studies can be performed to demonstrate the intravenous and oral pharmacokinetic behavior of a Formula I compound.

### A. Methods

A pharmacokinetic study is conducted with mice (Charles river) (N=3). The animals have free access to water and standard laboratory chow under a controlled 12 hour light-dark cycle. After at least one week of an acclimation period, the mice are administrated a dose of 5 mg/kg of the Formula I compound intravenously or 10 mg/kg orally. For both intravenous and oral administration, the compound is dissolved in saline. After intravenous administration, blood samples are collected at, for instance, 10 min, 30 min, 1, 2, 4, 8 and 24 hours. For oral gavage, blood samples are collected at, for instance, 15 min, 30 min 1, 2, 4, 8 and 24 hours. The blood samples are collected in polythene tubes with heparin sodium 100 unit/mL and centrifuged at 6000×g for 10 min to obtain plasma, which is stored at −20° C. until analysis.

The Formula I compound concentration in mouse plasma is quantitated using an LC/MS/MS (API 4000, AB SCIEX). Briefly, 50 μl mouse plasma is added to 100 μl acetonitrile with internal standard (Diltiazem 10 ng/ml). The mixture ias vortexed and centrifuged, and 5 μl supernatant is injected into LC/MS/MS for analysis. The separation is performed on, for instance, a Waters C18 column (2.1×50 mm, 3.5 μM particle size). The mobile phase consists of 0.2% pentafluoropropionic acid in water: acetonitrile by gradient elution. A Sciex API 4000 mass spectroscopy system equipped with an electrospray source in the positive-ion multiple reaction monitoring (MRM) mode is used for detection. The MRM transitions monitored for the Formula I compound and, for instance, diltiazem may be m/z 204.4/84.2 and m/z 415/178, respectively. The quantitation can range from 25 to 1000 ng/ml.

### B. Results—Intravenous Pharmacokinetics

Intravenous pharmacokinetics data for the Formula compound can include Cₘₐₓ, Tₘₐₓ, AUCₜₒₜ, CL (clearance), Vₛₛ (volume of distribution) and T_{1/2} (half life).

### Example 7

### Inhibition of Glucose Uptake in 3T3-L1 Adipocytes

The effect of a compound of Formula I on glucose uptake impairment in adipocytes can be determined as follows.

### A. 3T3-L1 Cell Culture:

3T3-L1 Mouse Embryonic Fibroblasts, ATCC® CL-173™ are differentiated to adipocytes using chemically-induced differentiation according to the ATCC guidelines. Differentiated 3T3-L1 Adipocyte 4e⁶ cell are incubated overnight in glucose free media (RPMI ref. #11879 Gibco) containing 1% dialyzed fetal bovine serum (ref. #26400 Gibco) at 37° C. Cells are washed with and incubated for 1 h with glucose free media before compound exposure. Cells are incubated with and without increasing concentration (e.g., 0.1, 1, 10 and 100 μM) of a compound of Formula I for 12 h at 37° C., followed by 3 wash steps in glucose free phosphate buffer. Cells are then exposed to oxidative stress by incubating with 4HNE/H₂O₂ at 100 μM each for 4 h in glucose free media at 37° C. After incubation, the cells are washed steps and ³C₆-glucose uptake assay is performed as previously reported (Datta et al., Cell Cycle. 2016 September; 15(17):2288-98. doi: 10.1080/15384101.2016.1190054. (Epub 2016 May 31)).

### B. Glucose Uptake

Cells are washed with PBS, followed by addition of glucose free DMEM medium containing 20 mM of ¹³C₆ glucose, 10 mM, 2-fluorodeoxyglucose (2-FDG) and insulin 10 μM (Sigma 15500) incubated for 1 hour at 37° C. 2-FDG is an inhibitor of glucose metabolism, thus preventing glucose breakdown and allowing its accumulation to further be measure and quantified. Cells are harvested, washed three times with 1×PBS and the cell pellet is lysed in 10 μl-PER buffer incubated 10 min at 4° C. and 90 μl of buffer A (20 mM ammonium hydroxide and 20 mM ammonium acetate) is added for extraction of metabolites and further analysis.

### C. Metabolite Extraction

Proteins from the supernatant cell media or from the cell lysates are precipitated with 3 volumes of acetonitrile, and the liquid phase is concentrated in a speed-vacuum (Dietmair et al., Anal Biochem. 2010, 404:155-64). The dry pellet containing the metabolites is resuspended in buffer A and used to measure the metabolites.

### D. Multiple Reaction Monitoring (MRM)

The detection of the ¹³C₆ glucose is carried out using, for instance, a Waters Xevo™ triple quadrupole tandem mass spectrometer (Waters Corp., Manchester, UK). IntelliStart software is used for method development of ¹³C₆ glucose metabolite and the optimization of the mass spectrometer parameters for best metabolite transitions detection condition. The UPLC/MS/MS method for the detection of ¹³C₆ glucose metabolite is developed for a complete separation and identification of the metabolic extracts for in less than 6 minutes per sample including a rapid elution of compounds and subsequent MRM. This method is confirmed using the metabolite standards in an ACQUITY UPLC® (100 mm×2.1 mm, 1.7 μm) BEH Amide C18 column heated to 40° C. employing a flow rate of 0.4 mL/min and mobile phases A and B, 20 mM ammonium hydroxide and 20 mM ammonium acetate in acetonitrile, respectively, and with the following gradient: 0% B, 0-1 min; 0% B-50% B, 1-2.5 min; 50% B-90% B, 2.5-3.2 min; 90% B to 0% B, 3.2-4 min; total run time 5 min. Chromatographic and mass spectrum data were collected and analyzed with Waters MassLynx v4.1 software. Quantification is obtained using linear regression analysis of the peak area ratio analyte versus concentration. The ratios of the precursor and fragment ions allow an accurate quantification of all the target metabolites. Standard calibration curves are performed using for ¹³C₆ glucose metabolite at concentrations of 0.01, 0.05, 0.1, 0.5, 1, 5, 10, 50, and 100 μM.

The disclosures of each and every patent, patent application, GenBank record, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

We claim:

1. A method for increasing insulin sensitivity, reducing insulin resistance and/or preventing insulin resistance in a subject in need thereof comprising administering to the subject an effective amount of a compound according to Formula I, or pharmaceutically acceptable salt thereof: 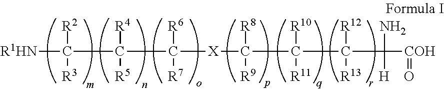
wherein:
X is selected from the group consisting of —O—, —S—, —NR¹⁴—O—, —O—NR¹⁴—, —NR¹⁴—NR¹⁵— and —S—S—;
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R², R³, R⁶, R⁷, R⁸, R⁹, R¹², and R¹³ are independently selected from the group consisting of hydrogen and —(C₁-C₆)alkyl;
R⁴ and R⁵ are independently selected from the group consisting of hydrogen, —(C₁-C₆)alkyl and —OH, provided that both R⁴ and R⁵ cannot be —OH;
R¹⁰ and R¹¹ are independently selected from the group consisting of hydrogen, —(C₁-C₆)alkyl and —OH, provided that both R¹⁰ and R¹¹ cannot be —OH;
R¹⁴ and R¹⁵ are independently selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl am selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
m is 1, 2 , 3 or 4;
n is 0, 1, 2, 3 or 4;
o is 0, 1, 2, 3 or 4;
p is 1, 2 , 3 or 4;
q is 0, 1, 2, 3 or 4; and
r is 0, 1, 2, 3 or 4.

2. The method according to claim 1 wherein X is selected from the group consisting of —O—, —S—, and —S—S—.

3. The method according to claim 2 wherein X is —O— and
(i) the sum of p+q+r is 1, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 3 or greater;
(ii). the sum of p+q+r is 2, the sum of m+n+o is 2, and R¹ cannot be ethyl; or
(iii) the sum of p+q+r is 2, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 3 or greater.

4. The method according to claim 2 wherein X is —S— and
(i) the sum of p+q+r is 1, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 5 or greater; or
(ii) the sum of p+q+r is 2, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 4 or greater.

5. The method according to claim 2 wherein X is —S—S and R1 is hydrogen, and wherein the sum of m+n+o is 2, 3 or 4 and/or the sum of p+q+r is 1, 2, 3 or 4.

6. The method according to claim 1 wherein X is selected from the group consisting of —NR¹⁴—O—, —O—NR¹⁴—, and —NR¹⁴—NR¹⁵—.

7. The method according to claim 6 wherein X is —NR¹⁴—O—, and R¹ and R¹⁴ are hydrogen, and wherein the sum of m+n+o is 2, 3 or 4 and/or the sum of p+q+r is 1, 2 or 3.

8. The method according to claim 6 wherein X is —O—NR¹⁴—, and R¹ and R¹⁴ are hydrogen, and wherein the sum of m+n+o is 2, 3 or 4 and/or the sum of p+q+r is 1, 2, 3 or 4.

9. The method according to claim 6 wherein X is —NR¹⁴—NR¹⁵—, R¹, R¹⁴ and R¹⁵ are hydrogen, and wherein the sum of m+n+o is 2, 3 or 4 and/or the sum of p+q+r is 1, 2, 3 or 4.

10. The method according to claim 2, wherein each of R² through R¹³ is independently selected from hydrogen and —(C₁-C₈)alkyl.

11. The method according to anyone of claims 1-10, wherein the compound of Formula I is the L-isomer substantially free of the D-isomer.

12. The method according to claim 1, wherein the compound of Formula I is (S)-2-amino-3-(3-aminopropoxy)propanoic acid.

13. A method of treating an insulin resistance disorder in a subject in need thereof comprising administering to the individual an effective amount of a compound according to Formula I, or pharmaceutically acceptable salt thereof: 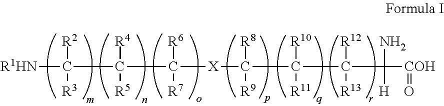
wherein:
X is selected from the group consisting of —O—, —S—, —NR¹⁴—O—, —NR¹⁴—, —NR¹⁴—NR¹⁵— and —S—S—;
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R², R³, R⁶, R⁷, R⁸, R⁹, R¹², and R¹³ are independently selected from the group consisting of hydrogen and —(C₁-C₆)alkyl;
R⁴ and R⁵ are independently selected from the group consisting of hydrogen, —(C₁-C₆)alkyl and —OH, provided that both R⁴ and R⁵ cannot be —OH;
R¹⁰ and R¹¹ are independently selected from the group consisting of hydrogen, —(C₁-C₆)alkyl and —OH, provided that both R¹⁰ and R¹¹ cannot be —OH;
R¹⁴ and R¹⁵ are independently selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
m is 1, 2 , 3 or 4;
n is 0, 1, 2, 3 or 4;
o is 0, 1, 2, 3 or 4;
p is 1, 2 , 3 or 4;
q is 0, 1, 2, 3 or 4; and
r is 0, 1, 2, 3 or 4.

14. The method according to claim 13, wherein the insulin resistance disorder is selected from the group consisting of diabetes, type 2 diabetes, pre-diabetes, obesity, metabolic syndrome, insulin resistance, insulin-resistance syndromes, syndrome X, high blood pressure, hypertension, high blood cholesterol, hyperlipidemia, dyslipidemia, atherosclerotic disease, hyperglycemia, hyperinsulinemia, hyperproinsulinemia, impaired glucose tolerance, delayed insulin release, coronary heart disease, angina pectoris, congestive heart failure, stroke, cognitive dysfunction, retinopathy, peripheral neuropathy, nephropathy, glomerulonephritis, glomerulosclerosis, nephrotic syndrome, hypertensive nephrosclerosis, endometrial cancer, breast cancer, prostate cancer, colon cancer, complications of pregnancy, menstrual irregularities, infertility, irregular ovulation, polycystic ovarian syndrome (PCOS), lipodystrophy, cholesterol related disorders, gout, obstructive sleep apnea, osteoarthritis, and osteoporosis.

15. A compound according to Formula I, or pharmaceutically acceptable salt thereof: 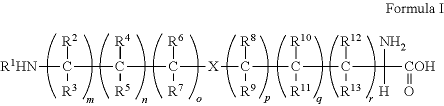
wherein:
X is selected from the group consisting of —O—, —S—, —NR¹⁴—O—, —O—NR¹⁴—, —NR¹⁴—NR¹⁵— and —S—S—;
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R², R³, R⁶, R⁷, R⁸, R⁹, R¹², and R¹³ are independently selected from the group consisting of hydrogen and —(C₁-C₆)alkyl;
R⁴ and R⁵ are independently selected from the group consisting of hydrogen, —(C₁-C₆)alkyl and —OH, provided that both R⁴ and R⁵ cannot be —OH;
R¹⁰ and R¹¹ are independently selected from the group consisting of hydrogen, —(C₁-C₆)alkyl and —OH, provided that both R¹⁰ and R¹¹ cannot be —OH;
R¹⁴ and R¹⁵ are independently selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
m is 1, 2 , 3 or 4;
n is 0, 1, 2, 3 or 4;
o is 0, 1, 2, 3 or 4;
p is 1, 2, 3 or 4;
q is 0, 1, 2, 3 or 4; and
r is 0, 1, 2, 3 or 4.

16. The compound according to claim 15 wherein X is selected from the group consisting of —O—, —S—, and —S—S—.

17. The compound according to claim 17 wherein X is —O— and
(i) the sum of p+q+r is 1, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 3 or greater;
(ii). the sum of p+q+r is 2, the sum of m+n+o is 2, and R¹ cannot be ethyl; or
(iii) the sum of p+q+r is 2, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 3 or greater.

18. The compound according to claim 17 wherein X is —S— and
(i) the sum of p+q+r is 1, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 5 or greater; or
(ii) the sum of p+q+r is 2, the definition of R¹, R², R³, R⁴, R⁵, R⁶, R⁷, R⁸, R⁹, R¹⁰, R¹¹, R¹², R¹³ and R¹⁴ are all H, and the sum of m+n+o is 4 or greater.

19. The method according to claim 17 wherein X is —S—S and R¹ is hydrogen, and wherein the sum of m+n+o is 2, 3 or 4 and/or the sum of p+q+r is 1, 2, 3 or 4.

20. The compound according to claim 16 wherein X is selected from the group consisting of —NR¹⁴—O—, —O—NR¹⁴—, and —NR¹⁴—NR¹⁵—.

21. The compound according to claim 20 wherein X is —NR¹⁴—O—, and R¹ and R¹⁴ are hydrogen, and wherein the sum of m+n+o is 2, 3 or 4 and/or the sum of p+q+r is 1, 2 or 3.

22. The compound according to claim 20 wherein X is —O—NR¹⁴—, and R¹ and R¹⁴ are hydrogen, and wherein the sum of m+n+o is 2, 3 or 4 and/or the sum of p+q+r is 1, 2, 3 or 4.

23. The compound according to claim 20 wherein X is —NR¹⁴—NR¹⁵—, R¹, R¹⁴ and R¹⁵ are hydrogen, and wherein the sum of m+n+o is 2, 3 or 4 and/or the sum of p+q+r is 1, 2, 3 or 4.

24. The compound according to claim 16, wherein each of R² through R¹³ is independently selected from hydrogen and —(C₁-C₈)alkyl.

24. The compound according to any one of claims 16-23, wherein the compound of Formula I is the L-isomer substantially free of the D-isomer.

25. The compound to claim 16, wherein the compound of Formula I is (S)-2-amino-3-(3-aminopropoxy)propanoic acid.


SEQUENCE LISTING (2 sequences)

SEQ ID NO: 1 - protein, 18 aa, Homo sapiens
  MISC_FEATURE (1)..(18): fragment of GLUT4 (amino acids 247-264)
LTGWADVSGVLAELKDEK

SEQ ID NO: 2 - protein, 18 aa, Artificial Sequence
  Adduct synthetically prepared
  MOD_RES (18)..(18): GLUT4-K264-NHE-adduct 4-NHE adduct linked to terminal lysine (corresponds to lysine 264 of GLUT4)
LTGWADVSGVLAELKDEK